SEQUENCE LISTING (25 sequences)

SEQ ID NO: 1 - DNA, 68 bp, Tobacco mosaic virus
gtatttttacaacaattaccaacaacaacaaacaacaaacaacattacaattactatttacaattaca

SEQ ID NO: 2 - DNA, 143 bp, Tobacco etch virus
aaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagca

SEQ ID NO: 3 - DNA, 36 bp, Alfalfa mosaic virus
gtttttatttttaattttctttcaaatacttccatc

SEQ ID NO: 4 - DNA, 84 bp, Nicotiana tabacum
atttaactcagtattcagaaacaacaaaagttcttctctacataaaattttcctattttagtgatcagtgaaggaaatcaagaa

SEQ ID NO: 5 - DNA, 101 bp, Oryza sativa
gaattccaagcaacgaactgcgagtgattcaagaaaaaagaaaacctgagctttcgatctctacggagtggtttcttgttctttgaaaaagagggggatta

SEQ ID NO: 6 - DNA, 58 bp, Arabidopsis thaliana
tacatcacaatcacacaaaactaacaaaagatcaaaagcaagttcttcactgttgata

SEQ ID NO: 7 - DNA, 107 bp, Zea mays
acaggctcatctcgctttggatcgattggtttcgtaactggtgagggactgagggtctcggagtggattgatttgggattctgttcgaagatttgcggaggggggca

SEQ ID NO: 8 - DNA, 206 bp, Zea mays
tctagactcccggcgaacactcccctccgccttcttatgctcatcccctccgcttccgaagcacaacatttcaaccagaaacactagccgaagcaaatccattccacaagcacctggtgggatcatctcatcatcagaaaccaagagagagattccgtgtccgcttgttgtagtagattgtgaggactgaggaccgagaagcagcc

SEQ ID NO: 9 - DNA, 107 bp, Zea mays
tctctcgcctgagaaaaaaaatccacgaaccaatttctcagcaaccagcagcacgacctgtgagggttcgaaggaagtagcagtgttttttgttcctagaggaagag

SEQ ID NO: 10 - DNA, 96 bp, Petunia x hybrida
cagaaaaatttgctacattgtttcacaaacttcaaatattattcatttatttgtcagctttcaaactctttgtttcttgtttgttgattgagaata

SEQ ID NO: 11 - DNA, 72 bp, Glycine max
tacacagaaacattcgcaaaaacaaaatcccagtatcaaaattcttctctttttttcatatttcgcaaagac

SEQ ID NO: 12 - DNA, 654 bp, Cestrum yellow leaf curling virus
gtttaattactggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcaggtggccaggaagaagaaaagccaagacgacgaaaacaggtaagagctaagctttctcatctcaaagatgattcttgatgatttttgtctccaccgtccgtataggatcactgaattgataaatatcatatggtttgtataaaacccgatatttaaatctgtatcattctgtttgaataaaacttgatactttgttggagtcgtttgtaaaaacataaacc

SEQ ID NO: 13 - DNA, 6 bp, Glycine max
aaaaaa

SEQ ID NO: 14 - DNA, 57 bp, Glycine max
atggccaagctcgtgttctctctttgcttccttcttttctccggatgctgcttcgct

SEQ ID NO: 15 - DNA, 2148 bp, Artificial
  Soy optimized endoglucanase
atggctactcaaggtgctcttgattctgctgttaccgctcttcagtctgctattaccaccttctctggtgctagacaagatggtgctaagacctctggattcacctctgctcaagtgaccgctcttatcaactctgctaaggctgacaaagagggtgttaggacttctgctaacggtgatgatgtttccccagttgagtactgggttaactcttctgtgctcggagctttcaacgctgctattaccgctttggagaacgcttctggacagtcagctatcgatgctgcttacctcgctcttattcaggctggaaagaccttcaacgatgctaagaggcatggaactactccagataggaccgctctcaacaacgctattaccgcagctgttaacgctaagaacggtgttcaaaccgctgctgataaggatcaggcttctcttggatcttcttgggctactggtgctcaattcaacgctctcaacaccgcaattgattctgctaccgccgttaagaacaacgctaacgctaccaaggcttctgttgatactgctgctgcttctcttaacgctgctatcgctactttcactaccgctgtgactaacaacggaccaggaactcaaaccttcagggatattaccgctgctcaactcgttgctgagatcaagattggatggaaccttggaaactcccttgatgctcacaacggattcccagctaacccaaccgttgatcagatggaaagaggatggggaaacccagctactaccaaggctaacatcaccgctcttaagaacgctggattcaacgctattaggattccagtgtcctggactaaggctgcttctggtgctccaaactacaccattaggaccgattggatgactagggtgaaagagatcgttaactacgctgtggacaacgacatgtacatcatccttaacacccaccatgatgaggatgtgctcaccttcatgaactctaacgctgctgctggaaaggctgctttccaaaagttgtgggagcaaatcgctgctgctttcaaggattacaacgagaagctcattttcgagggacttaacgagccaagaaccccaggatcttctaacgaatggaacggtggaactgatgaagagaggaacaacctcaactcctactacccaatcttcgtgaacactgtgagatcttccggtggaaacaacggaaaaaggattctcatgatcaacccatacgctgcttctatggaagctgtggctatgaacgctcttactctcccagctgattctgctgctaacaagctcattgtgtccttccattcctaccagccatacaacttcgctcttaacaaggactcctccattaacacctggtcctcttcctcttctggtgatacctctccaattaccggaccaatcgacaggtactacaacaagttcgtgtcccaaggtattccagtgatcattggagagttcggcgctatgaacaagaacaacgaggctgttagagcacaatgggctgagtactacgtttcttacgctcagtccaagggtattaagtgcttctggtgggataacggtgttacttctggatctggcgagcttttcggacttcttaacaggaccaacaacaccttcacctacaacgctcttctcaacggaatgatgtctggaactggtggtactgttccaactccaccaactcctccagctactccaactcctcctactaccattaccggaaaccttggaacttaccagttcggaacccaagaggatggtgtgtctcctaactacactcaagctgtgtgggagctttctggaacaaaccttaccactgctaagactaccggtgctaagttggttcttgtgttcaccactgctccaaacgcttctatgcactttgtttggcagggaccagctaattctctttggtggaacgagaaagagattctcggaaacaccggaaacccatctgctactggtgttacctggaactctggaactaagacccttaccattccacttaccgctaactccgtgaaggattactctgttttcaccgcccaaccatcccttaggatcatcatcgcttactacaacggtggaaacgtgaacgatctcggaattgtgtctgctaacctcactcaatga

SEQ ID NO: 16 - DNA, 18 bp, Artificial
  Soy optimized ER retention signal
tctgagaaggatgagctt

SEQ ID NO: 17 - DNA, 70 bp, Cauliflower mosaic virus
cttagtatgtatttgtatttgtaaaatacttctatcaataaaatttctaattcctaaaaccaaaatccag

SEQ ID NO: 18 - DNA, 144 bp, Tobacco etch virus
taaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagca

SEQ ID NO: 19 - DNA, 122 bp, Maize necrotic streak virus
agatatcgacctgcctgaccaggctgagattgcgctagccggcgtagttggtatctctcgcgcaagcgggtttgaaggtgcggcctatcttaggggggtaaattgtaacttcgcacaaaggc

SEQ ID NO: 20 - DNA, 2778 bp, Zea mays
tagaggcaacccaagataggtgaaagataagcttcctttgtcacaattgaatattcgtgcaaggtggtccaactattattttgagatgtttattgagaccattgaggacctttgagtaattaactctcaacctagtagaaattcgttaccaactgggttgcataggatttcatgattaacagtgtgtttggtttagctgtgagttttctcctatgaaaagactgttgtgagaacaaaaagttgaaaatcgtttagttcaaactgttgtgagttatccactgtaaacaaattgtatattgtttatatacactatgtttaactatatctcttaatcaatatatacaattaaaaaactaaattcacatttgtgttcctaatattttttacaaataaatcattgttcgattccatttgtaatattttttattaaaattgtttttatttcatttattataaacacttaattgttttaatcctattttagtttcaatttattgtatctatttattaatataacgaacttcgataagaaacaaaagcaaggtcaaggtgttttttcagggctagtttgggagtccaaaaattggagggggttagaggggctaaaatctcattcttattcaaaattgaataaggaggggattttagcccctctaatcatcttcagttttgtggctcccaaactagccctcaaagtagatgtggaaaagttgaaccccttttattcagcttctagaagcaggtttgaaaaatagaaccaaacaaaccctaaaagtgtgtgaatttttaacaggtaatggcaggttaattattcacatctctttggtcatgtttaagaggctgaaaatagatcaattgcaagaacaaatagcagagtggataggggtggggaggggtcgtctccctatctgacctctctcctgcattggattgcctttctccgtactctatttaaaagtacaaatgaggtgccggattgatggagtgatatataagtttgatgtgtttttcacatacgtgacaagtattattgaaagagaacagttgcattgctactgtttggatatgggaaaactgagaattgtatcatgcgatggccgatcagttctttacttagctcgatgtaattaatgcacaatgttgatagtatgtcgaggatctagagatgtaatggtgttaggacacgtggttagctactaatataaatgtaaggtcaaaattcgatggtttattttctattttcaattacctagcattatctcatttctaattgtgtgataacaaatgcattagaccataattctgtaaatacgtacatttaagcacacagtctatattttaaaattcttctttttgtgtggatatcccaacccaaatccacctctctcctcaatccgtgtatcttcaccgctgccaagtgccaacaacacatcgcatcgtgcaaatctttgttggtttgtgcacggtcggcgccaatggaggagacacctgtacggtgcccttggtagaacaacatccttatccctatatgtatggtgcctttcgtagaatggcaccccttatccctacaatagccatgtatgcataccaagaattaaatatactttttcttgaaccacaataatttattatagcggcacttcttgttctggttgaacacttatttggaacaataaaatcccgagttcctaaccacaggttcactttttttccttatcctcctaggaaactaaattttaaattcataaatttaattgaaatgttaatgaaaacaaaaaaattatctacaaagacgactcttagccacagccgcctcactgcaccctcaaccacatcctgcaaacagacaccctcgccacatccctccagattcttccctccgatgcagcctacttgctaacagacgccctctccacatcctgcaaagcattcctccaaattcttgcgatcccccgaatccagcattaactgctaagggacgccctctccacatcctgctacccaattagccaacggaataacacaagaaggcaggtgagcagtgacaaagcacgtcaacagcaccgagccaagccaaaaaggagcaaggaggagcaagcccaagccgcagccgcagctctccaggtccccttgcgattgccgccagcagtagcagacacccctctccacatcccctccggccgctaacagcagcaagccaagccaaaaagaagcctcagccacagccggttccgttgcggttaccgccgatcacatgcccaaggccgcgcctttccaaacgccgagggccgcccgttcccgtgcacagccacacacacacccgcccgccaacgactccccatccctatttgaacccacccgcgcactgcattgatcaccaatcgcatcgcagcagcacgagcagcacgccgtgccgctccaaccgtctcgcttccctgcttagcttcccgccgcgccttggcgtcgaccaaggcacccggccccggcgagaagcaccactccatcgacgcgcagctccgtcagctggtcccaggcaaggtctccgaggacgacaagctcatcgagtacgaagcgctgctcgtcgaccgcttcctcaacatcctccaggacctccacgggcccagccttcgcgaatttgtaactaaccaccgccgcggcccatttcttcttcgaccggttgccgcctgcgcgcggcactggtcgtgtcgtgtgctcgctcgtctccctccggtgcttactactgtaatccttgcaggtccaggagtgctacgaggtaaa

SEQ ID NO: 21 - DNA, 57 bp, Zea mays
atgcgcgtgctgctcgttgcgctggccctgctggctctggccgcttctgcgacctct

SEQ ID NO: 22 - DNA, 6 bp, Zea mays
taaacc

SEQ ID NO: 23 - DNA, 2148 bp, Artificial
  Maize optimized endoglucanase
atggccacccagggcgctctggacagcgccgtgactgcgctccaatccgctatcaccaccttcagcggtgctcgccaggatggcgccaagaccagcggcttcaccagcgcccaggtgaccgccctgatcaacagcgccaaggccgacaaggagggcgtccgcacttctgctaacggcgacgacgtgtccccggtggagtactgggtgaacagcagcgtgctgggcgcgttcaatgctgccatcaccgccctcgagaacgccagcggccagagcgctatcgacgccgcctacctggctctcatccaggccggcaagacgttcaatgacgcgaagcgccacggcaccaccccagacaggaccgccctcaacaacgcgatcaccgccgctgtgaacgccaagaacggcgtccagaccgccgctgacaaggaccaggccagcctgggcagcagctgggctaccggcgctcagttcaacgccctgaacaccgccatcgacagcgccaccgccgtgaagaacaacgccaacgccaccaaggccagcgtggacaccgccgctgccagcctgaacgccgcgatcgccaccttcaccaccgccgtcaccaacaacggcccaggcacccagaccttccgcgacatcaccgctgcccagctcgtggccgagatcaagatcggctggaacctgggcaacagcctggacgcccacaacggcttcccggccaacccaaccgtggaccagatggaacgcggctggggcaacccagccaccaccaaggcgaacatcaccgcgctgaagaacgccggcttcaacgccatccgcatcccggtgtcctggaccaaggccgccagcggcgctccgaactacaccatccgcaccgactggatgacccgcgtgaaggagatcgtcaactacgccgtggacaacgacatgtacatcatcctgaacacccaccacgacgaggacgtgctgaccttcatgaacagcaacgccgctgccggcaaggccgccttccagaagctgtgggagcagatcgccgctgccttcaaggactacaacgagaagctgatcttcgagggcctgaacgagccaaggaccccgggcagcagcaacgagtggaacggcggcaccgacgaggagcgcaacaacctgaacagctactacccgatcttcgtgaacactgtgcgcagcagcggcggcaacaacggcaagcgcatcctgatgatcaacccctacgccgccagcatggaagccgtggccatgaacgccctgaccctgccagccgactccgccgccaacaagctgatcgtgtccttccacagctaccagccgtacaacttcgccctgaacaaggacagcagcatcaacacctggtccagcagcagctccggcgacaccagcccaatcaccggcccgatcgaccgctactacaacaagttcgtgtcccagggcatcccggtgatcatcggcgagttcggcgccatgaacaagaacaacgaggctgtgcgcgcccagtgggccgagtactacgtgtcctacgcccagagcaagggcatcaagtgcttctggtgggacaacggcgtgaccagcggctctggcgagctgttcggcctgctgaaccgcaccaacaacaccttcacctacaacgccctgctgaacggcatgatgagcggcaccggcggcactgtgcccacgccacccacccctccggccaccccaaccccgccaaccaccatcaccggcaacctgggcacctaccagttcggcacccaggaggacggcgtttcccccaactacacccaggctgtgtgggagctgtccggcacgaatctgacgaccgccaagaccacgggcgccaagctggtgctggtgttcaccaccgcgccgaacgccagcatgcactttgtgtggcagggtccagctaatagcctgtggtggaacgagaaggagatcctgggcaacaccggcaacccatctgctacgggcgttacctggaacagcggcaccaagaccctgaccatcccgctgaccgccaacagcgtgaaggactactccgtgttcaccgcccagccgagcctgcgcatcatcatcgcctactacaacggcggcaacgtgaacgacctgggcatcgtgtccgccaacctgacccagtga

SEQ ID NO: 24 - DNA, 18 bp, Artificial
  Maize optimized ER retention signal
agcgagaaggacgagctg

SEQ ID NO: 25 - DNA, 1002 bp, Zea mays
gcggcttctcttcactcacctgcagagtgcaccgcaataatcagcttccggatggtggcgttttgtcagttttggatggaaatgccgaactggcagcgtctgttttccctatgcatatgtaatttcctgcctctttatattcactcttgttgtcaagtccaagtggaaaatcttggcatattatacatattgtaataataaacatcgtacaatctgcatgctgttttgtaataattaattaatatcccagcccattggatggacttgtttaccaaggtgttacttcagtcaccctcttttagttgtgctaaacagtttctgattgatatttttttattagagtaacctagtgcatttacttaagagaaatgatatctagtggcactagtgattagtttgcaaggttgagaacttgttactcgctcctagaggttaacactagcaagtgattggagcttagggtttttcttgaatttcactagaaaaaatataaactagtatatcatgatatgcacttaagtctttttagtgttatctaccgacactcaaaaaggctttcttgctactcatttctcttactcctaaagcaaaaaaaaaatagccaaatgaccctccctctaacaataatcataatgaaatctcacctctcttttaggtgcaatatttttgtgggagtgggtctttttgggtgactgaggggctctaggaaggggatcagtagagatatctagcaaggtgtcaagtgtattcctgagatggttaggttttgaacaccacacatgtttctgaggaggggctctcataagctccttaggcactccatctctcacaataggggtggcagatttgggaggagtgagcttgacatgtttggggtggatgaaggtttctctgaaggttttaggccactacactcaccaaccttaccaacacaagtgacactcccatccttagcagcaaagcctaaccccgttcccccagttcccctcttgaactaactga

(19) United States
(12) Patent Application Publication
(10) Pub. No.: US 2012/0185969 A1
(43) Pub. Date: Jul. 19, 2012

(54) STACKING OF TRANSLATIONAL ENHANCER ELEMENTS TO INCREASE POLYPEPTIDE EXPRESSION IN PLANTS

(75) Inventors: Andrew DeBrecht, Cary, NC (US); Kasimalai Azhakanandam, Durham, NC (US)

(73) Assignee: Syngenta Participations AG

(21) Appl. No.: 13/393,846

(22) PCT Filed: Sep. 2, 2010
(86) PCT No.: PCT/US10/47693

§ 371 Date: Apr. 4, 2012

Related U.S. Application Data
(60) Provisional application No. 61/240,118, filed on Sep. 4, 2009.

(51) Int. Cl. A01H 5/00; C12N 5/10; A01H 5/10; C12N 15/82

(52) U.S. Cl. 800/298; 435/320.1; 435/419

(57) ABSTRACT

Compositions and methods for increasing expression of a polypeptide of interest in a plant or plant part thereof are provided. Compositions of the invention are polynucleotide constructs comprising (a) at least one translational enhancer element derived from a virus tandemly stacked with at least one translational enhancer element derived from a cellular gene, and (b) an operably linked polynucleotide encoding a polypeptide of interest. Expression cassettes, vectors, and transgenic plants and plant parts comprising these polynucleotide constructs are also provided. Methods for increasing expression of a polypeptide of interest in a plant or plant part thereof utilizing the polynucleotide constructs and expression cassettes of the invention are also provided.

### CROSS REFERENCE TO RELATED APPLICATIONS

This application claims priority from U.S. Provisional Application No. 61/240,118, filed Sep. 4, 2009.

### FIELD OF THE INVENTION

The invention relates generally to plant molecular biology, particularly to compositions and methods for increasing expression of transgenes in plants.

### REFERENCE TO SEQUENCE LISTING SUBMITTED ELECTRONICALLY

The official copy of the Sequence Listing is submitted electronically via EFS-Web as an ASCII formatted Sequence Listing with a file named “42473 SEQ Listing_ST25.txt,” created on Sep. 2, 2010, having a size of 17 kb and is filed concurrently with the specification. The Sequence Listing contained in this ASCII formatted document is part of the specification and is herein incorporated by reference in its entirety.

### BACKGROUND

Advances in plant genetic engineering have enabled the production of plants with agronomically desirable traits and an ability to serve as viable recombinant protein expression systems. These advances depend upon a proper expression of recombinant polynucleotide constructs encoding one or more polypeptides of interest in a transgenic plant into which they are introduced. Previous work provides a number of regulatory elements, such as promoters, introns and translational leaders, that are useful to effect expression of such recombinant polynucleotide constructs in transgenic plants. However, many previously identified regulatory elements fail to provide levels of recombinant protein expression required to fully realize the intended benefits of the introduced expression of selected genes in transgenic plants. Thus, there is still a great need for novel regulatory elements and combinations of regulatory elements that are capable of directing high levels of expression of desired polypeptides in plants.

### BRIEF SUMMARY

Compositions and methods for increasing expression of a polypeptide of interest in a plant or plant part thereof are provided. Compositions of the invention are polynucleotide constructs comprising (a) at least one translational enhancer element derived from a virus tandemly stacked with at least one translational enhancer element derived from a cellular gene, and (b) an operably linked polynucleotide encoding a polypeptide of interest. Expression cassettes, vectors, and transgenic plants and plant parts comprising these polynucleotide constructs also are provided. One or more of the translational enhancer elements may be heterologous to the polypeptide of interest. Heterologous refers to a sequence not derived from the leader sequence (5′UTR) of the expressed polypeptide of interest.

Methods of the invention comprise introducing into a plant or plant part thereof a polynucleotide construct of the invention operably linked to a promoter that is functional in a plant cell. When cultured under conditions suitable for expression of a polynucleotide construct of the invention, the tandemly stacked translational enhancer elements provide for greater efficiency in translation of the related mRNA transcript. The methods of the present invention thus provide for increased expression of a polypeptide of interest in a plant or plant part thereof.

The following embodiments are encompassed by the present invention.

1. A polynucleotide construct comprising (a) at least one translational enhancer element derived from a virus tandemly stacked with at least one translational enhancer element derived from a cellular gene, and (b) an operably linked polynucleotide encoding a polypeptide of interest.

2. The polynucleotide construct of embodiment 1, wherein said virus is a plant virus.

3. The polynucleotide construct of embodiment 2, wherein said virus is an

RNA virus.

4. The polynucleotide construct of embodiment 3, wherein said virus is a member of the Group IV (+)ssRNA viruses, and wherein said translational enhancer element derived from said virus comprises the leader sequence (5′ UTR) of said virus.

5. The polynucleotide construct of embodiment 4, wherein said virus is a member of the genus Tobamovirus or is a member of a family selected from the group consisting of the Potyviridae, Bromoviridae, and Tombusviridae.

6. The polynucleotide construct of embodiment 5, wherein said virus is selected from the group consisting of tobacco mosaic virus (TMV), tobacco etch virus (TEV), alfalfa mosaic virus (AMV), and maize necrotic streak virus (MNeSV).

7. The polynucleotide construct of embodiment 6, wherein said virus is TMV, and wherein said translational enhancer element derived from said TMV comprises the leader sequence set forth in SEQ ID NO:1 or a functional fragment or variant thereof, wherein said variant has at least 95% sequence identity to the sequence set forth in SEQ ID NO:1.

8. The polynucleotide construct of embodiment 6, wherein said virus is TEV, and wherein said translational enhancer element derived from said TEV comprises the leader sequence set forth in SEQ ID NO:2 or SEQ ID NO:18, or a functional fragment or variant thereof, wherein said variant has at least 95% sequence identity to the sequence set forth in SEQ ID NO:2 or SEQ ID NO:18.

9. The polynucleotide construct of embodiment 6, wherein said virus is AMV or MNeSV, and wherein said translational enhancer element derived from said AMV or said MNeSV comprises the leader sequence set forth in SEQ ID NO:3 or SEQ ID NO:19, respectively, or a functional fragment or variant thereof, wherein said variant has at least 95% sequence identity to the sequence set forth in SEQ ID NO:3 or SEQ ID NO:19.

10. The polynucleotide construct of any one of embodiments 1-9, wherein said cellular gene is a stress response gene.

11. The polynucleotide construct of embodiment 10, wherein said cellular stress response gene is selected from the group consisting of an alcohol dehydrogenase gene and a heat shock protein gene.

12. The polynucleotide construct of embodiment 11, wherein said alcohol dehydrogenase gene is from a monocot plant or a dicot plant.

13. The polynucleotide construct of embodiment 12, wherein said alcohol dehydrogenase gene is from tobacco, rice, Arabidopsis, soy or maize.

14. The polynucleotide construct of embodiment 13, wherein said translational enhancer element derived from said cellular gene comprises the tobacco alcohol dehydrogenase leader sequence set forth in SEQ ID NO: 4, or a functional fragment or variant thereof, wherein said variant has at least 95% sequence identity to the sequence set forth in SEQ ID NO:4.

15. The polynucleotide construct of embodiment 13, wherein said translational enhancer element derived from said cellular gene comprises the rice alcohol dehydrogenase leader sequence set forth in SEQ ID NO:5, or a functional fragment or variant thereof, wherein said variant has at least 95% sequence identity to the sequence set forth in SEQ ID NO:5.

16. The polynucleotide construct of embodiment 13, wherein said translational enhancer element derived from said cellular gene comprises the Arabidopsis alcohol dehydrogenase leader sequence set forth in SEQ ID NO: 6, or a functional fragment or variant thereof, wherein said variant has at least 95% sequence identity to the sequence set forth in SEQ ID NO:6.

17. The polynucleotide construct of embodiment 13, wherein said translational enhancer element derived from said cellular gene comprises the maize alcohol dehydrogenase leader sequence set forth in SEQ ID NO:7, or a functional fragment or variant thereof, wherein said variant has at least 95% sequence identity to the sequence set forth in SEQ ID NO:7.

18. The polynucleotide construct of embodiment 11, wherein said heat shock protein gene is from a monocot plant or a dicot plant.

19. The polynucleotide construct of embodiment 18, wherein said heat shock protein gene is from maize, soybean, or petunia.

20. The polynucleotide construct of embodiment 19, wherein said translational enhancer element derived from said cellular gene comprises the maize heat shock protein 101 leader sequence set forth in SEQ ID NO:5 or a functional fragment or variant thereof, wherein said variant has at least 95% sequence identity to the sequence set forth in SEQ ID NO:5.

21. The polynucleotide construct of any one of embodiments 1-20, wherein said operably linked polynucleotide encodes a polypeptide that imparts a phenotype selected from the group consisting of insect resistance, disease resistance, herbicide resistance, abiotic stress resistance, a modified enzyme expression profile, a modified oil content, and a modified nutrient content.

22. An expression cassette comprising the polynucleotide construct of any one of embodiments 1-21.

23. The expression cassette of embodiment 22, wherein said polynucleotide construct is operably linked to a promoter that is functional in a plant cell, and wherein said promoter is selected from the group consisting of a constitutive promoter, an inducible promoter, and a tissue-specific promoter.

24. A plant comprising the polynucleotide construct of embodiment 1 or the expression cassette of embodiment 22.

25. The plant of embodiment 24, wherein said plant is a monocot plant or a dicot plant.

26. The plant of embodiment 25, wherein said plant is selected from the group consisting of rice, barley, potato, sweet potato, canola, sunflower, rye, oats, wheat, corn, soybean, sugar beet, tobacco, Miscanthus grass, Switch grass, safflower, trees, cotton, cassaya, tomato, sorghum, alfalfa, and sugarcane.

27. The plant of any one of embodiments 24-26, wherein said polynucleotide construct or said expression cassette is stably integrated into the genome of the plant, and wherein said polynucleotide construct is operably linked to a promoter that is functional in a plant cell.

28. A cell of the plant of any one of embodiments 24-27, wherein said cell comprises said polynucleotide construct or said expression cassette stably integrated into its genome, and wherein said polynucleotide construct is operably linked to a promoter that is functional in a plant cell.

29. Seed of the plant of any one of embodiments 24-28, wherein said seed comprises said polynucleotide construct or said expression cassette stably integrated into its genome, and wherein said polynucleotide construct is operably linked to a promoter that is functional in a plant cell.

30. A method for increasing expression of a polypeptide of interest in a plant or plant part thereof, said method comprising introducing into said plant or said plant part a polynucleotide construct that is operably linked to a promoter that is functional in a plant cell, wherein said polynucleotide construct comprises (a) at least one translational enhancer element derived from a virus tandemly stacked with at least one translational enhancer element derived from a cellular gene, and (b) an operably linked polynucleotide encoding said polypeptide of interest.

31. The method of embodiment 30, wherein said virus is a plant virus.

32. The method of embodiment 31, wherein said virus is an RNA virus.

33. The method of embodiment 32, wherein said virus is a member of the Group IV (+)ssRNA viruses, and wherein said translational enhancer element derived from said virus comprises the leader sequence (5′ UTR) of said virus.

34. The method of embodiment 33, wherein said virus is a member of the genus Tobamovirus or is a member of a family selected from the group consisting of the Potyviridae, Bromoviridae, and Tombusviridae.

35. The method of embodiment 34, wherein said virus is selected from the group consisting of tobacco mosaic virus (TMV), tobacco etch virus (TEV), alfalfa mosaic virus (AMV), and maize necrotic streak virus (MNeSv).

36. The method of embodiment 35, wherein said virus is TMV, and wherein said translational enhancer element derived from said TMV comprises the leader sequence set forth in SEQ ID NO:1 or a functional fragment or variant thereof, wherein said variant has at least 95% sequence identity to the sequence set forth in SEQ ID NO:1.

37. The method of embodiment 35, wherein said virus is TEV, and wherein said translational enhancer element derived from said TEV comprises the leader sequence set forth in SEQ ID NO:2 or SEQ ID NO:18, or a functional fragment or variant thereof, wherein said variant has at least 95% sequence identity to the sequence set forth in SEQ ID NO:2 or SEQ ID NO:18.

38. The method of embodiment 35, wherein said virus is AMV or MNeSV, and wherein said translational enhancer element derived from said AMV or said MNeSV comprises the leader sequence set forth in SEQ ID NO:3 or SEQ ID NO:19, respectively, or a functional fragment or variant thereof, wherein said variant has at least 95% sequence identity to the sequence set forth in SEQ ID NO:3 or SEQ ID NO:19.

39. The method of any one of embodiments 30-38, wherein said cellular gene is a stress response gene.

40. The method of embodiment 39, wherein said cellular stress response gene is selected from the group consisting of an alcohol dehydrogenase gene and a heat shock protein gene.

41. The method of embodiment 40, wherein said alcohol dehydrogenase gene is from a monocot plant or a dicot plant.

42. The method of embodiment 41, wherein said alcohol dehydrogenase gene is from tobacco, rice, Arabidopsis, soy or maize.

43. The method of embodiment 42, wherein said translational enhancer element derived from said cellular gene comprises the tobacco alcohol dehydrogenase leader sequence set forth in SEQ ID NO:4 or a functional fragment or variant thereof, wherein said variant has at least 95% sequence identity to the sequence set forth in SEQ ID NO:4.

44. The method of embodiment 42, wherein said translational enhancer element derived from said cellular gene comprises the rice alcohol dehydrogenase leader sequence set forth in SEQ ID NO: 5, or a functional fragment or variant thereof, wherein said variant has at least 95% sequence identity to the sequence set forth in SEQ ID NO:5.

45. The method of embodiment 42, wherein said translational enhancer element derived from said cellular gene comprises the Arabidopsis alcohol dehydrogenase leader sequence set forth in SEQ ID NO: 6, or a functional fragment or variant thereof, wherein said variant has at least 95% sequence identity to the sequence set forth in SEQ ID NO: 6.

46. The method of embodiment 42, wherein said translational enhancer element derived from said cellular gene comprises the maize alcohol dehydrogenase leader sequence set forth in SEQ ID NO: 7, or a functional fragment or variant thereof, wherein said variant has at least 95% sequence identity to the sequence set forth in SEQ ID NO: 7.

47. The method of embodiment 40, wherein said heat shock protein gene is from maize, soybean, or petunia.

48. The method of embodiment 47, wherein said translational enhancer element derived from said cellular gene comprises the maize heat shock protein 101 leader sequence set forth in SEQ ID NO: 5 or a functional fragment or variant thereof, wherein said variant has at least 95% sequence identity to the sequence set forth in SEQ ID NO: 5.

49. The method of any one of embodiments 30-48, wherein said operably linked polynucleotide encodes a polypeptide that imparts a phenotype selected from the group consisting of insect resistance, disease resistance, herbicide resistance, abiotic stress resistance, a modified enzyme expression profile, a modified oil content, and a modified nutrient content.

50. The method of any one of embodiments 30-49, wherein said polynucleotide construct is operably linked to a promoter selected from the group consisting of a constitutive promoter, an inducible promoter, and a tissue-specific promoter.

51. The method of any one of embodiments 30-50, wherein said plant is a monocot plant or a dicot plant.

52. The method of embodiment 51, wherein said plant is selected from the group consisting of rice, barley, potato, sweet potato, canola, sunflower, rye, oats, wheat, corn, soybean, sugar beet, tobacco, Miscanthus grass, Switch grass, safflower, trees, cotton, cassaya, tomato, sorghum, alfalfa and sugarcane.

53. The method of any one of embodiments 30-52, wherein said polynucleotide construct is stably integrated into the genome of the plant or plant part thereof.

54. The method of any one of embodiments 30-53, wherein expression of said polypeptide of interest in said plant or plant part thereof is increased by at least 2-fold when compared to expression of said polypeptide of interest in a wild-type plant or plant part thereof, or in a control plant or plant part thereof.

55. The method of embodiment 54, wherein expression of said polypeptide of interest in said plant or plant part thereof is increased by at least 4-fold when compared to expression of said polypeptide of interest in said wild-type plant or plant part thereof, or in said control plant or plant part thereof.

56. A polynucleotide construct comprising (a) the translational enhancer element derived from the tobacco mosaic virus (TMV) 5′ UTR set forth in SEQ ID NO:1, tandemly stacked with the tobacco alcohol dehydrogenase 5′ UTR set forth in SEQ ID NO:4, and (b) an operably linked polynucleotide encoding a polypeptide of interest, wherein said polynucleotide construct is operably linked to a promoter that is functional in a plant cell.

57. A method for increasing expression of a polypeptide of interest in a plant or plant part thereof, said method comprising introducing a polynucleotide construct comprising (a) the translational enhancer element derived from the tobacco mosaic virus (TMV) 5′ UTR set forth in SEQ ID NO: 1, tandemly stacked with the tobacco alcohol dehydrogenase 5′ UTR set forth in SEQ ID NO:4, and (b) an operably linked polynucleotide encoding a polypeptide of interest, wherein said polynucleotide construct is operably linked to a promoter that is functional in a plant cell.

59. A polynucleotide construct comprising (a) the translational enhancer element derived from the alfalfa mosaic virus (AMV) 5′ UTR set forth in SEQ ID NO: 3, tandemly stacked with the tobacco alcohol dehydrogenase 5′ UTR set forth in SEQ ID NO: 4, and (b) an operably linked polynucleotide encoding a polypeptide of interest, wherein said polynucleotide construct is operably linked to a promoter that is functional in a plant cell.

60. A method for increasing the expression of a polypeptide of interest in a plant or plant part thereof, said method comprising introducing a polynucleotide construct comprising (a) the translational enhancer element derived from the alfalfa (AMV) 5′ UTR set forth in SEQ ID NO: 3, tandemly stacked with the tobacco alcohol dehydrogenase 5′ UTR set forth in SEQ ID NO: 4, and (b) an operably linked polynucleotide encoding a polypeptide of interest, wherein said polynucleotide construct is operably linked to a promoter that is functional in a plant cell.

61. A polynucleotide construct comprising (a) the translational enhancer element derived from the tobacco mosaic virus (TMV) 5′ UTR set forth in SEQ ID NO: 1, tandemly stacked with the Zea mays alcohol dehydrogenase 5′ UTR set forth in SEQ ID NO: 7, and (b) an operably linked polynucleotide encoding a polypeptide of interest, wherein said polynucleotide construct is operably linked to a promoter that is functional in a plant cell.

62. A method for increasing the expression of a polypeptide of interest in a plant or plant part thereof, said method comprising introducing a polynucleotide construct comprising (a) the translational enhancer element derived from the tobacco mosaic virus (TMV) 5′ UTR set forth in SEQ ID NO: 1, tandemly stacked with the Zea mays alcohol dehydrogenase 5′ UTR set forth in SEQ ID NO: 7, and (b) an operably linked polynucleotide encoding a polypeptide of interest, wherein said polynucleotide construct is operably linked to a promoter that is functional in a plant cell.

63. A polynucleotide construct comprising (a) the translational enhancer element derived from the tobacco etch virus (TEV) 5′ UTR set forth in SEQ ID NO: 18, tandemly stacked with the tobacco alcohol dehydrogenase 5′ UTR set forth in SEQ ID NO:4, and (b) an operably linked polynucleotide encoding a polypeptide of interest, wherein said polynucleotide construct is operably linked to a promoter that is functional in a plant cell.

64. A method for increasing the expression of a polypeptide of interest in a plant or plant part thereof, said method comprising introducing a polynucleotide construct comprising (a) the translational enhancer element derived from the tobacco etch virus (TEV) 5′ UTR set forth in SEQ ID NO: 18, tandemly stacked with the tobacco alcohol dehydrogenase 5′ UTR set forth in SEQ ID NO: 4, and (b) an operably linked polynucleotide encoding a polypeptide of interest, wherein said polynucleotide construct is operably linked to a promoter that is functional in a plant cell.

### BRIEF DESCRIPTION OF THE DRAWINGS

FIG. 1 shows the effect of the tobacco mosaic virus (TMV) f 5′ leader and the tobacco alcohol dehydrogenase (ADH) 5′ leader, alone and tandemly stacked within the expression cassette, on endoglucanase (EG) expression. Enhanced expression was observed when the viral 5′ leader (i.e., 52) was positioned upstream of the cellular 5′ leader (i.e., 5′-ADH). Four plants were examined for each of the five constructs tested (not including the control vector), with each bar representing an average activity from an individual plant (x-axis is EG activity (μmol/min/mg of total soluble protein); y-axis is each individual plant).

FIGS. 2A-B also show the effect of the tobacco mosaic virus (TMV) Ω 5′ leader, also referred to as “Ω,” and the tobacco alcohol dehydrogenase (ADH) 5′ leader, alone and tandemly stacked within the expression cassette, on endoglucanase (EG) expression. FIG. 2A shows endoglucanase expression on a leaf fresh weight basis, and FIG. 2B shows endoglucanase expression on a total soluble protein basis. Enhanced expression was observed when the viral 5′ leader sequence (i.e., a) was positioned upstream of the cellular 5′ leader sequence (i.e., 5′-ADH). Three to four plants were examined for each of the five constructs tested (x-axis is EG activity (μmol/min/g or mg); y-axis is each construct).

### BRIEF DESCRIPTION OF THE SEQUENCE LISTING

SEQ ID NO: 1 is a tobacco mosaic virus (TMV) 5′ UTR.

SEQ ID NO: 2 is a tobacco etch virus (TEV) 5′ UTR.

SEQ ID NO: 3 is an alfalfa mosaic virus (AMV) 5′ UTR.

SEQ ID NO: 4 is a tobacco alcohol dehydrogenase (ADH) 5′ UTR.

SEQ ID NO: 5 is a rice alcohol dehydrogenase (ADH) 5′ UTR.

SEQ ID NO: 6 is an Arabidopsis alcohol dehydrogenase (ADH) 5′ UTR.

SEQ ID NO: 7 is a maize alcohol dehydrogenase (ADH) 5′ UTR.

SEQ ID NO: 8 is a maize heat shock protein 101 (HSP101) 5′ UTR.

SEQ ID NO: 9 is a maize heat shock protein 70 (HSP70) 5′ UTR.

SEQ ID NO: 10 is a petunia heat shock protein 101 (HSP101) 5′ UTR.

SEQ ID NO: 11 a soybean heat shock protein 17.9 (HSP17.9) 5′ UTR.

SEQ ID NO: 12 is a cestrum yellow leaf curling virus promoter.

SEQ ID NO: 13 is a soybean Kozak sequence.

SEQ ID NO: 14 is a soybean glycinin seed protein CDS.

SEQ ID NO: 15 is a soybean optimized endoglucanase CDS.

SEQ ID NO: 16 is a soybean optimized ER retention signal.

SEQ ID NO: 17 is a cauliflower mosaic virus (CMV) terminator.

SEQ ID NO: 18 is a tobacco etch virus (TEV) 5′ UTR.

SEQ ID NO: 19 is a maize necrotic streak virus 5′ UTR.

SEQ ID NO: 20 is a maize PEPC promoter.

SEQ ID NO: 21 is a maize gamma zein signal sequence.

SEQ ID NO: 22 is a maize Kozak sequence.

SEQ ID NO: 23 is a maize optimized endoglucanase CDS.

SEQ ID NO: 24 is a maize optimized ER retention signal.

SEQ ID NO: 25 is a maize PEPC terminator.

### DETAILED DESCRIPTION OF THE INVENTION

The present invention is directed to compositions and methods for increasing expression of a polypeptide of interest in plants or plant parts thereof. Compositions include polynucleotide constructs comprising tandemly stacked viral and cellular translational enhancer elements positioned upstream of (i.e., at the 5′ end) and operably linked with a polynucleotide encoding a polypeptide of interest. When incorporated into an expression cassette with an operably linked promoter of interest, the tandemly stacked viral and cellular translational enhancer elements provide for increased efficiency of translation of the related mRNA transcript, thereby increasing expression of the encoded polypeptide of interest when compared to the level of expression of that polypeptide from a polynucleotide construct that lacks the operably linked, tandemly stacked viral and cellular translational enhancer elements. As such, compositions and methods are described herein for making and using transgenic plants having increased expression of a polypeptide of interest, where the transgenic plants comprise an expression cassette comprising a polynucleotide construct with at least two tandemly stacked translational enhancer elements operably linked to a polynucleotide encoding a polypeptide of interest, where at least one translational enhancer element is of viral origin and at least one translational enhancer element is of cellular origin. The invention includes the use of heterologous enhancers.

The compositions and methods described herein find use in applications where increased protein production in a plant or plant part thereof is warranted. Such applications include, but are not limited to, genetic manipulation of metabolic pathways to improve agronomic performance of plants, e.g., increased disease resistance, herbicide resistance, nutrient utilization, and environmental stress resistance; to alter agronomic characteristics, e.g., modifications in starch, oil, fatty acid, or protein content/composition to enhance animal and human nutrition, improve digestibility, and/or improve processing traits; to develop modifications, such as male sterility, senescence, and the like; and to introduce transgene expression of pharmaceuticals, industrial enzymes, and the like.

### Polynucleotide Constructs

The present invention is directed to polynucleotide constructs that provide for increased expression of a polypeptide of interest in a plant or plant part thereof. As used herein, “polynucleotide construct” means a polymer of nucleotides, such as deoxyribonucleotides, ribonucleotides, or modified forms thereof, in the form of an individual fragment or as a component of a larger construct, in a single-stranded or in a double-stranded form. The polynucleotides include sense and antisense polynucleotide sequences of DNA or RNA as appropriate to the goals of the methods practiced according to the invention. The DNA or RNA molecules may be complementary DNA (cDNA), genomic DNA, synthesized DNA, or a hybrid thereof, or an RNA molecule such as mRNA, including untranslated and translated regions. As used herein, “DNA construct,” “gene construct,” “polynucleotide,” and “polynucleotide construct” mean both DNA and RNA molecules.

The polynucleotide constructs of the invention comprise (a) at least one viral translational enhancer element tandemly stacked with at least one cellular translational enhancer element, and (b) an operably linked polynucleotide encoding a polypeptide of interest. As used herein, “operably linked,” when referring to a first nucleic acid sequence that is operably linked with a second nucleic acid sequence, means a situation when the first nucleic acid sequence is placed in a functional relationship with the second nucleic acid sequence. For instance, a promoter is operably linked to a coding sequence if the promoter effects the transcription of the coding sequence. Likewise, the coding sequence of a signal peptide is operably linked to the coding sequence of a polypeptide if the signal peptide effects the extracellular secretion of that polypeptide. Generally, operably linked nucleic acid sequences are contiguous and, where necessary to join two protein coding regions, the open reading frames are aligned. In the context of the polynucleotide constructs of the invention, the tandemly stacked translational enhancer elements are operably linked to a polynucleotide encoding a polypeptide of interest, and thus are functionally related in that the tandemly stacked translational enhancer elements increase translation of the related mRNA transcript, thereby increasing expression of the encoded polypeptide of interest. While not intending to be bound to any particular theory, translation may be increased due to the effects of the tandemly stacked translational enhancer elements on the processing of the primary transcript to mRNA, mRNA stability, translation efficiency, or any combination thereof.

As used herein, “translational enhancer element” means a polynucleotide that enhances (i.e., increases) translation and is positioned upstream (i.e., in the 5′ direction on the same nucleic acid sequence) of a polynucleotide encoding a polypeptide of interest. A translational enhancer element is transcribed into RNA as part of a fully processed mRNA transcript, but is not translated, and facilitates (i.e., promotes) translation of the downstream mRNA transcript, thereby increasing expression of the encoded polypeptide of interest. Translational enhancer element of the invention may include heterologous sequences. Heterologous sequences are sequences not derived from the leader sequence of the expressed gene of interest.

While not intending to be bound to any particular theory, translational enhancer elements are believed to recruit trans-acting factors such as RNA binding proteins (e.g., heat-shock proteins and other translation initiation factors such as eukaryotic initiation factor (eIF-1 to 4), ribosomal subunits, translation elongation factors (for example, eukaryotic elongation factor (eEF-1 or -2), and the like, which ultimately enhance translation of an mRNA transcript. The term “translational enhancer element” expressly excludes cis-acting transcriptional enhancing elements such as promoters, TATA boxes, CAAT boxes, and the like. Thus, the tandemly stacked translational enhancer elements within the polynucleotide constructs of the invention are to be contrasted with tandemly stacked cis-acting transcriptional elements known in the art, the latter of which are used in polynucleotide constructs to increase transcription of an operably linked, transcribable polynucleotide of interest, for example, a polynucleotide encoding a polypeptide or inhibitory RNA molecule (e.g., interfering RNA, such as hairpin RNAi).

Translational enhancer elements include, but are not limited to, translation leader sequences (i.e., 5′ UTR), and elements or domains positioned therein, that are capable of increasing expression of a polypeptide encoded by an operably linked polynucleotide via their ability to enhance translation of the resultant mRNA transcript. As used herein, “translation leader sequence,” “5′ UTR,” “leader sequence” or “5′ leader” means a polynucleotide derived or isolated from an upstream regulatory region of genomic DNA (i.e., genes) or mRNA that starts at the transcription start site and ends just before the first translation initiation codon (usually ATG in the DNA sequence, AUG in the mRNA transcript) of a coding sequence. Those of skill in the art also refer to translation leader sequences as “5′ untranslated leader sequences” or “5′ non-translated leader sequences.”

For purposes of the present invention, the term “translational enhancer element” is not to be construed as meaning solely a Kozak sequence. By “Kozak sequence,” “Kozak consensus sequence,” or “Kozak consensus” is intended a short consensus sequence that surrounds the initiating start codon (AUG) within a mRNA. Based on 699 vertebrate mRNAs, Kozak proposed (GCC)GCC(A/G)CCAUGG as the consensus sequence for the context of the functional AUG codon (underlined in the consensus sequence) within the mRNA (see, for example, Kozak et al. (1987) Nucleic Acids Res. 15(20):8125-8148).

The Kozak sequence is recognized by the ribosome as the translation start site, from which point a protein is coded by that mRNA molecule, and plays a major role in the initiation of the translation process (see, for example, De Angioletti et al. (2004) Br. J. Haematol. 124(2):224-231; Kozak (1984) Nature 308:241-246; Kozak (1986) Cell 44(2):283-292)). For viral mRNAs, the Kozak sequence surrounding the initiating AUG is generally ACCAUGG, with the most consistent position located three nucleotides before the initiation codon (AUG) and almost always in an adenine (A) nucleotide. The mRNAs of higher plants have an AC-rich consensus sequence, CAA(A/C)AAUGGCG. Between the two major groups of angiosperms, the context of the AUG codon in dicot mRNAs is AAA(A/C)AAUGGCU, which is similar to the higher-plant consensus but monocot mRNAs have C(A/C)(A/G)(A/C)CAUGGCG as a consensus, which exhibits an overall similarity with the vertebrate consensus proposed by Kozak (see, for example, Joshi et al. (1997) Plant Mol. Biol. 35:993-1001). Although the ribosome requires the Kozak sequence, or a variation of this sequence, to initiate translation, the Kozak sequence is distinguishable from the ribosomal binding site (RBS) (i.e., the 5′ cap of a messenger RNA or an Internal Ribosome Entry Site (IRES)). Thus, where a portion or fragment of a 5′ UTR serves as a translational enhancer element for use in the present invention, that portion may comprise a suitable Kozak sequence but will not consist solely of that Kozak sequence.

Translational enhancer elements may be isolated from a genomic copy of a gene. Thus, for example, a translation leader sequence, or elements or domains therein, may be isolated from the untranslated 5′ region (5′ UTR). Alternatively, translational enhancer elements, such as translation leader sequences and functional elements or domains therein that enhance translation, may be synthetically produced or manipulated non-coding DNA elements. Translational enhancer elements useful for practicing the present invention are of viral or cellular origin. The length of any given translational enhancer element will vary, but is typically less than about 250 base pairs (bp) in length, less than about 225 bp in length, less than about 200 bp in length, less than about 175 bp in length, or less than about 150 bp, less than about 125 bp, less than about 100 bp, less than about 75 bp, less than about 50 bp, or less than about 25 bp in length, and typically is at least about 10 bp in length. In some embodiments, the length of any given translational enhancer element is about 10 bp to about 250 bp, including, for example, about 10 bp, 15, bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 105 bp, 110 bp, 115 bp, 120 bp, 125 bp, 130 bp, 135 bp, 140 bp, 145 bp, 150 bp, 155 bp, 160 bp, 165 bp, 170 bp, 175 bp, 180 bp, 185 bp, 190 bp, 195 bp, 200 bp, 205 bp, 210 bp, 215 bp, 220 bp, 225 bp, 230 bp, 235 bp, 240 bp, 245 bp, 250 bp, or any such length between about 10 bp and about 250 bp. It is not necessary that each translational enhancer element that is to be tandemly stacked with one or more additional translational enhancer elements must be the same length, and in fact, typically the translational enhancer elements are of a different length, depending upon the length of the native translational enhancer element or fragment thereof that is to be included within a polynucleotide construct of the invention.

As used herein, “tandemly stacked” means that the at least one viral translational enhancer element and the at least one cellular translational enhancer element are positioned sequentially or consecutively (i.e., one behind the other, in that order) in the polynucleotide construct of the invention. This is in contrast to the viral and cellular translational enhancer elements being positioned alone (i.e., singly) within the polynucleotide construct, or being positioned randomly with respect to each other in the polynucleotide construct (e.g., random positioning would be exemplified where the viral translational enhancer element is located upstream of a polypeptide coding sequence, and the cellular translational enhancer element is located downstream of the coding sequence and/or within the coding sequence). Furthermore, the at least one viral translational enhancer element is positioned upstream (i.e., at the 5′ end) of the at least one cellular enhancer element.

Although the viral and translational enhancer elements are preferably immediately adjacent to one another (i.e., no intervening nucleotides positioned between the 3′ end of the viral translational enhancer element and the 5′ end of the cellular translational enhancer element), it is recognized that the tandemly stacked translational enhancer elements can comprise a linker sequence positioned between their respective 3′ and 5′ ends within the polynucleotide construct. When present, the linker sequence can be a single nucleotide up to as many as 30 nucleotides, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. Thus, in some embodiments the tandemly stacked translational enhancer elements can comprise a linker sequence that is 1 to about 30 nucleotides, 1 to about 25 nucleotides, 1 to about 20 nucleotides, 1 to about 15 nucleotides, 1 to about 10 nucleotides, or 1 to about 5 nucleotides in length. In other embodiments, the tandemly stacked translational enhancer elements can comprise a linker sequence that is at least 2 nucleotides up to about 30 nucleotides, at least 5 nucleotides up to about 30 nucleotides, at least 10 nucleotides up to about 30 nucleotides, at least 15 nucleotides up to about 30 nucleotides, at least 20 nucleotides up to about 30 nucleotides, at least 25 nucleotides up to about 30 nucleotides, at least 2 nucleotides up to about 25 nucleotides, at least 2 nucleotides up to about 20 nucleotides, at least 2 nucleotides up to about 15 nucleotides, at least 2 nucleotides up to about 10 nucleotides, at least 2 nucleotides up to about 5 nucleotides, at least 5 nucleotides up to about 30 nucleotides, at least 5 nucleotides up to about 25 nucleotides, at least 5 nucleotides up to about 20 nucleotides, at least 5 nucleotides up to about 15 nucleotides, at least 5 nucleotides up to about 10 nucleotides, at least 10 nucleotides up to about 30 nucleotides, at least 10 nucleotides up to about 25 nucleotides, at least 10 nucleotides up to about 20 nucleotides, at least 10 nucleotides up to about 15 nucleotides, at least 15 nucleotides up to about 30 nucleotides, at least 15 nucleotides up to about 25 nucleotides, at least 15 nucleotides up to about 20 nucleotides, or at least 20 nucleotides up to about 25 nucleotides in length.

Of particular interest to the present invention are translational enhancer elements of viral origin and cellular origin. In some embodiments, the cellular translational enhancer elements are of eukaryotic cellular origin, including, for example, animal or plant cellular origin. Thus, the polynucleotide constructs of the invention preferably comprise (a) at least one translational enhancer element derived from a virus tandemly stacked with at least one translational enhancer element derived from a cellular gene, and (b) an operably linked polynucleotide encoding a polypeptide of interest. As used herein, “derived from” means that the translational enhancer sequence is either obtained from (e.g., isolated from) a naturally occurring nucleic acid sequence of a virus or cellular gene, or is designed (i.e., engineered) from a naturally occurring nucleic acid sequence of a virus or cellular gene.

Any known virus can serve as a source of a translational enhancer element for use in practicing the present invention. Thus, for example, the virus can be from a varying range of hosts, including, for example, bacteria, fungi, plants, animals, and insects. The virus can be a DNA virus or an RNA virus. By “DNA virus” is intended a virus that has DNA as its genetic material and replicates using a DNA-dependent DNA polymerase. The nucleic acid of a DNA virus can be double-stranded DNA (dsDNA) or single-stranded DNA (ssDNA). By “RNA virus” is intended a virus that has RNA as its genetic material. The nucleic acid of an RNA virus can be single-stranded (ssRNA) or double-stranded RNA (dsRNA). RNA viruses are further classified according to the sense or polarity of their RNA into negative-sense (−) and positive-sense (+), or ambisense RNA viruses. Positive-sense viral RNA is identical to viral mRNA and thus can be immediately translated by the host cell. Negative-sense viral RNA is complementary to mRNA and thus must be converted to positive-sense RNA by an RNA polymerase before translation. Ambisense RNA viruses resemble negative-sense RNA viruses, except they also translate genes from the positive strand. Thus, the viral translational enhancer element may be derived from any viral source.

In some embodiments, the viral translational enhancer element is derived from a plant virus, i.e., the host organism for the virus is a plant. In some of these embodiments, the translational enhancer element is from an RNA plant virus. Any RNA plant virus can serve as a source of the viral translational enhancer element.

RNA plant viruses of interest include, but are not limited to, members of the Group IV viruses in accordance with the Baltimore classification system for viruses. The Baltimore classification system places viruses into one of seven groups depending on a combination of their nucleic acid (DNA or RNA), strandedness (single-stranded or double-stranded), sense (i.e., polarity), and method of replication. The Baltimore Group IV viruses possess positive-sense (+) single-stranded (ss) RNA genomes (referred to as Group IV (+)ssRNA viruses). Examples of Group IV (+)ssRNA plant viruses include, but are not limited to, plant viruses of the family Bromoviridae, Potyviridae, and Tombusviridae, as well as plant viruses of the Tobamovirus genus. Exemplary members of the Bromoviridae family include, but are not limited to, viruses of the genus Alfamovirus, genus Anulavrius, genus Ilarvirus, genus Bromovirus, genus Cucumovirus, and genus Oleavirus. Exemplary members of the Potyviridae family include, but are not limited to, viruses of the genus Potyvirus, genus Rymovirus, genus Bymovirus, genus Macluravirus, genus Ipomovirus, and genus Tritimovirus. Exemplary members of the Tombusviridae include, but are not limited to, viruses of the genus Tombusvirus, genus Carmovirus, genus Necrovirus, genus Dianthovirus, genus Machlomovirus, genus Avenavirus, and genus Panicovirus. However, the classification of plant viruses is under review, and this listing of viral families and genus members is not intended to limit the scope of the plant viral source of the viral translational enhancer elements for use in practice of the invention, as any suitable translational enhancer element derived from an RNA plant virus can be used to practice the present invention in the manner set forth herein.

In some embodiments, the viral translational enhancer element is derived from a Group IV (+) ssRNA virus selected from the group consisting of Alfalfa mosaic virus (AMV; Bromoviridae family), tobacco streak virus (TSV; Bromoviridae family), brome mosaic virus (BMV; Bromoviridae family), cucumber mosaic virus (CMV; Bromoviridae family), tobacco etch virus (TEV; Potyviridae family), potato virus Y (PVY; Potyviridae family), ryegrass mosaic virus (Potyviridae family), barley yellow mosaic virus (Potyviridae family), maclura mosaic virus (Potyviridae family), sweet potato mild mottle virus (SPMMV; Potyviridae family), wheat streak mosaic virus (WSMV; Potyviridae family), maize necrotic streak virus (MNeSV; Tombusviridae family), tomato bushy stunt virus (TBSV; Tombusviridae family); carnation ringspot virus (CRSV; Tombusviridae family); red clover necrotic mosaic virus (Tombusviridae family), sweet clover necrotic mosaic virus (Tombusviridae family), tobacco mosaic virus (TMV; Tobamovirus genus), U2-tobacco mosaic virus (T2MV; Tobamovirus genus), tomato mosaic virus (ToMV; Tobamovirus genus), cucumber green mottle mosaic virus (CGMMV; Tobamovirus genus), cucumber virus 4 (CV4; Tobamovirus genus), Frangipani virus (FV; Tobamovirus genus), odontoglosum ringspot virus (ORSV; Tobamovirus genus), ribgrass mosaic virus (HRV; Tobamovirus genus), sun hemp mosaic virus (SHMV; Tobamovirus genus), beet necrotic yellow vein virus (BNYVV; tentatively assigned to Tobamovirus genus), Nicotiana velutina mosaic virus (NVMV; tentatively assigned to Tobamovirus genus), peanut clump virus (PCV; tentatively assigned to Tobamovirus genus), potato mop-top virus (PMTV; tentatively assigned to Tobamovirus genus), and soil-borne wheat mosaic virus (SBWMV; tentatively assigned to Tobamovirus genus). The foregoing list of Group IV (+)ssRNA viruses is merely illustrative of the viral sources from which a translational enhancer element for use in the present invention can be derived, and is not intended to limit the scope of the invention.

Translational enhancer elements of viral origin are well known in the art. For example, the polynucleotide constructs of the invention may comprise at least one viral translational enhancer element tandemly stacked with at least one cellular translational enhancer element, where the viral translational enhancer element may comprise a picornavirus translation leader sequence, e.g., Encephalomyocarditis (EMCV) 5′ leader (Elroy-Stein et al. (1989) Proc. Natl. Acid. Sci. USA 86:6126-6130); a potyvirus translation leader sequence, e.g., tobacco etch virus (TEV) 5′ leader (Allison et al. (1986) Virology 154:9-20; and Gallie et al. (1995) Gene 165:233-238); maize dwarf mosaic virus (MDMV) 5′ leader (Allison et al. (1986) Virology 154:9-20); potato etch virus (PEV) 5′ leader (Tomashevskaya et al. (1993) J. Gen. Virol. 74:2717-2724); translation leader of potato virus S genomic RNA (Turner et al. (1999) Archives Virol. 144(7):1451-1461); translation leader sequence from the coat protein mRNA of alfalfa mosaic virus (AMV RNA 4 5′ leader) (Jobling et al. (1987) Nature 325:622-625; U.S. Pat. No. 6,037,527); tobacco mosaic virus (TMV) 5′ leader (Gallie et al. (1987) Nucleic Acids Res. 15:3257-3273; Gallie et al. (1988) Nucleic Acids Res. 16:883-893; Gallie et al. (1992) Nucleic Acids Res. 20:4631-4638; U.S. Pat. No. 5,489,527); maize necrotic streak virus (MNeSv) 5′ leader (Louie et al. (2000) Plant Dis. 84:1133-1139; SEQ ID NO:19); and maize chlorotic mottle virus (MCMV) 5′ leader (Lommel et al. (1991) Virology 81:382-385).

It is recognized that the translational enhancer elements of RNA viral origin (for example, the TMV 5′ leader) can be used as such to prepare a polynucleotide construct of the invention by ligating them upstream of an appropriate mRNA transcript complementary to a polynucleotide comprising the cellular translational enhancer element and operably linked polynucleotide encoding the polypeptide of interest, using, e.g., T₄ RNA ligase. Preferably the polynucleotide constructs of the invention are designed for use in an expression cassette or expression vector, in which case, the translational enhancer elements of RNA viral origin are used in the form of complementary DNA. The cDNA of an RNA viral translational enhancer element can be obtained using conventional methods known to those of skill in the art. In some embodiments, the cDNA of the RNA viral translational enhancer element is chemically synthesized for incorporation into the expression cassette or expression vector. Thus, for purposes of the present invention, a polynucleotide construct comprising a translational enhancer element of viral origin encompasses the presence of the RNA or cDNA sequence of that translational enhancer element.

Translational enhancer elements of cellular genes are also known in the art, and may be derived from any cellular gene. Of particular interest herein are translational enhancer elements of cellular genes that are highly expressed within a particular host cell. While not being bound by any theory or mechanism of action, translational enhancer elements from highly expressed genes may function at a higher level. Highly expressed genes may be defined as those that, when expressed, represent at least 10% of the mRNA of the cell. Alternatively, highly expressed genes may encode for proteins that when expressed, represent greater than 1% of the total soluble protein of a specific cell or tissue type. In some embodiments, highly expressed genes may encode for proteins that when expressed, represent greater than 1% of the total soluble protein of a specific cell or tissue type and may not represent at least 10% of the mRNA of the cell.

In some embodiments, the translational enhancer elements are derived from cellular stress response genes, including, for example, animal or plant cellular stress response genes. As used herein, “stress response genes” means genes that are upregulated by external conditions that adversely affect growth, development, or productivity of an organism. Such stresses can be either biotic (imposed by other organisms) or abiotic (arising from an excess or deficit in the physical or chemical environment, such as a shortage or excess of solar energy, nutrient depletion, soil salinity, high (heat and drought) and low (cold and freezing) temperature, oxidative stress, or pollution (e.g., heavy metals). Examples of stress response genes include, but are not limited to, alcohol dehydrogenase genes, abscisic acid (ABA) genes, and heat shock protein genes (see also, U.S. Pat. No. 7,109,033; Seki et al. (2001) Plant Cell 13:61-72; Seki et al. (2002) Funct. Integr. Genomic. 2:282-291; and Seki et al. (2002) Plant J. 31:279-292, each of which is incorporated herein by reference.

As such, the polynucleotide constructs of the invention can comprise at least one viral translational enhancer element tandemly stacked with at least one cellular translational enhancer element, where the cellular translational enhancer element may comprise a translation leader sequence from an alcohol dehydrogenase (ADH) gene, e.g., the translation leader sequence of the tobacco ADH gene (NtADH 5′ leader; Satoh et al. (2004) J. Bioscience Bioengineering 98(1):1-8), the translation leader sequence for the rice ADH2 gene (OsADH2 5′ leader; Sugio et al. (2008) J. Bioscience Bioengineering 105(3):300-302), the translation leader sequence for the Arabidopsis thaliana ADH gene (AtADH 5′ leader; Sugio et al. (2008) J. Bioscience Bioengineering 105(3):300-302), and the translation leader sequence for the maize ADH1 gene (ADH1 5′ leader); and a translation leader sequence from a heat shock protein (HSP) gene, for example, the translation leader sequence for the maize HSP101 gene (HSP101 5′ leader; Nieto-Sotelo et al. (1999) Gene 230:187-195), and the translation leader sequences for the petunia HSP70, soybean HSP17.9, and maize HSP70 genes see for example, U.S. Pat. Nos. 5,659,122 and 5,362,865, herein incorporated by reference in their entirety). Other suitable cellular translational enhancer elements include, but are not limited to, the translation leader sequence for the tobacco photosystem I gene psaDb (psaDb 5′ leader; Yamamoto et al. (1995) J. Biol. Chem. 270(21):12466-12470); Fed-1 5′ leader (Dickey (1992) EMBO J 11:2311-2317); and rubisco small subunit (RbcS) 5′ leader (Silverthorne et al. (1990) J. Plant. Mol. Biol. 15:49-58).

Thus, in some embodiments of the invention, the translational enhancer elements are translation leader sequences, including, but not limited to, the translation leader sequences described above. Although the translation leader sequences for use in the polynucleotide constructs of the invention may be the full-length, native (i.e., naturally occurring) 5′ UTR sequence, it is recognized that functional fragments and variants of these leader sequences may be used to practice the claimed invention. Thus, the native translation leader sequences described herein may be varied (e.g., by substitution, insertion, or deletion) or truncated (either at the 5′ end and/or the 3′ end) such that their function as a translational enhancer is modulated (i.e., increased or decreased) so long as that function is not destroyed, and the variant or truncated sequence retains the ability to increase expression of a polypeptide of interest when used in tandem with at least one other translational enhancer element. Identification of those regions within a native translation leader sequence that are amenable to alteration (i.e., substitution, insertion, deletion, or truncation) or those portions of a native translation leader that represent functional fragments is readily determined by one of skill in the art using, e.g., standard mutational analysis. See, e.g., Gallie et al. (1988) Nucleic Acids Res. 16(3):883-893. Accordingly, although the following discussion refers to polynucleotide constructs comprising full-length, native, translation leader sequences as the translational enhancer elements, each disclosed embodiment contemplates the use of functional variants and fragments of these translation leader sequences, where those variants and fragments are defined as set forth elsewhere herein.

In some embodiments of the invention, the polynucleotide constructs of the invention comprise (a) at least one copy of the tobacco mosaic virus translation leader sequence (TMV 5′ leader) tandemly stacked with at least one cellular translational enhancer element, and (b) an operably linked polynucleotide encoding a polypeptide of interest. The TMV 5′ leader, also referred to as Ω, is a 68-base pair (bp) sequence from the TMV genomic RNA (see, e.g., Gallie et al. (1987) Nucleic Acids Res. 15:8693-8711; Gallie et al. (1987) Nucleic Acids. Res. 15:3257-3273). The cDNA sequence for Ω is set forth in SEQ ID NO:1. The Ω leader sequence is highly structured: three copies of an eight-base (5′-ACAAUUAC-3′) direct repeat and one copy of a 27-base poly(CAA) region (located between the 5′ eight-base direct repeat and the other two copies of this repeat) comprise 72% of the leader (Gallie (1996) “Post-transcriptional Control in Transgenic Gene Design,” in Transgenic Plants: A Production System for Industrial and Pharmaceutical Proteins, Chapters 1-3, ed. Owen and Pen (Wiley, Hoboken, N.J.). Although the 5′ untranslated leader sequences from four different strains of TMV vary in length, they all contain roughly equivalent repeats and a poly(CAA) sequence (Kukla et al. (1979) Eur. J. Biochem 98:61-66; and Goelet et al. (1982) Proc. Natl. Acad. Sci. USA 79:5818-5822).

As noted elsewhere herein, functional variants and fragments of the TMV 5′ leader may be used in the polynucleotide constructs of the invention and still provide for increased expression of a polypeptide of interest when tandemly stacked with at least one translational enhancer element of a cellular gene. Functional analysis of Ω has identified the poly(CAA) region as the primary element responsible for the enhancement of translation of an operably linked open-reading frame in vivo (Gallie and Walbot (1992) Nucleic Acids Res. 20:4631-4638; and Gallie et al. (1988) Nucleic Acids Res. 16:883-893). Functional variants and fragments of the Ω leader sequence are known in the art, and include, but are not limited to, for example, deletion mutants ΩΔ1 (lacking nt 2-9 of SEQ ID NO: 1), ΩΔ2 (lacking the first eight-base direct repeat, corresponding to nt 12-19 of SEQ ID NO: 1), ΩΔ3 (lacking nt 1-23 of the 27-bp poly(CAA) region, corresponding to nt 20-42 of SEQ ID NO: 1), ΩΔ4 (lacking the second eight-base direct repeat, corresponding to nt 47-54 of SEQ ID NO: 1), ΩΔ5 (lacking the third eight-base direct repeat, corresponding to nt 60-67 of SEQ ID NO: 1), and the variant sequences designated as ΩA,C→U (replacement of the poly(CAA) region with poly(U), and ΩA→C (single base substitution in the AUU sequence of the 5′ eight-base direct repeat, replacing AUU with CUU). Although the ΩΔ3 deletion mutant and the variant) Ω,C→U sequence are functional, preferably the poly(CAA) region is retained within a fragment or variant of the Ω leader sequence in order to maximize the increased translational efficiency provided by this translational enhancer element. See, e.g., Gallie et al. (1988), supra.

In other embodiments, the polynucleotide constructs of the invention comprise (a) at least one copy of the tobacco etch virus translation leader sequence (TEV 5′ leader) tandemly stacked with at least one cellular translational enhancer element, and (b) an operably linked polynucleotide encoding a polypeptide of interest. Tobacco etch virus (TEV) is a potyvirus, a member of the picornavirus supergroup of positive-strand RNA viruses that infects plants. The genomic RNA of TEV is a polyadenylated mRNA that naturally lacks a 5′ cap structure but that is nevertheless efficiently translated. The 143-base pair (bp) TEV 5′ leader (shown in SEQ ID NO: 2) is sufficient to confer cap-independent translation to an mRNA (Carrington and Freed (1990) J. Virology 64:1590-1597; Gallie (2001) J. Virology 75:12141-12152) and is functionally analogous to a cap in that it interacts with the poly(A) tail to promote translation (Gallie et al. (1995) Gene 165:233-238). Two centrally located cap-independent regulatory elements (CIREs) within the 143-bp TEV 5′ leader are required to direct cap-independent translation, and, when used as a single translation leader, both are required to interact functionally with the poly(A) tail to promote optimal translation (Niepel and Gallie (1999) J. Virology 73:9080-9088). These CIREs are positioned within nt 28-65 (CIRE-1) and nt 66-118 (CRIE-2) of SEQ ID NO: 2.

The functional TEV 5′ leader has been reported to be 144-bp in length (see Carrington and Freed (1990) supra), wherein the sequence is identical to that shown in SEQ ID NO: 2, but includes a thymine (t) nucleotide inserted prior to position 1 of SEQ ID NO: 2 (see the 144-bp sequence set forth in SEQ ID NO: 18). Thus, the 144-bp TEV 5′ leader has the following five (5) nucleotides at its 5′ end: 5′-taaat-3′, as opposed to the initial five nucleotides for the 143-bp TEV 5′ leader shown in SEQ ID NO: 2 (i.e., 5′-aaata-3′). The above description of the location of the CIREs, and the following discussion regarding variants and fragments of the TEV leader, is with respect to the 143-bp sequence shown in SEQ ID NO: 2. It is recognized that the nucleotide (nt) positions within the 143-bp sequence, as described for the CIREs above and variants and fragments below, can be identified within the 144-bp sequence, by adjusting the position to account for the single nucleotide insertion at the 5′ end of the 143-bp sequence shown in SEQ ID NO:2. Thus, for example, where CIRE-1 is positioned within nt 28-65 of SEQ ID NO: 2, the corresponding location within the 144-bp TEV 5′ leader is at nt 29-66 of SEQ ID NO:18. It is to be understood that the 144-bp TEV 5′ leader also can be used as the source of the viral translational enhancer element within the polynucleotide constructs of the invention.

Thus, variants and fragments of the TEV leader may be used in the polynucleotide constructs of the invention as long as they comprise at least one of these CIREs, preferably both of these CIREs. Functional fragments of the TEV leader are known in the art, and include, but are not limited to, the deletion mutants TEV₂₈₋₁₄₃ (lacking nt 1-27 of SEQ ID NO: 2), TEV₁₋₁₁₈ (lacking nt 119-143 of SEQ ID NO: 2), TEV₂₈₋₁₁₈(lacking nt 1-27 and 119-143 of SEQ ID NO:2), TEV₁₋₆₅(lacking nt 66-143 of SEQ ID NO: 2), TEV₆₆₋₁₄₃ (lacking nt 1-65 of SEQ ID NO: 2), TEV₂₈₋₆₅ (lacking nt 1-27 and 66-143 of SEQ ID NO:2), and TEV₆₆₋₁₁₈ (lacking nt 1-65 of SEQ ID NO: 2). See, e.g., Niepel and Gallie (1999), supra.

In yet other embodiments, the polynucleotide constructs of the invention comprise (a) at least one copy of the translation leader sequence from the coat protein (CP) mRNA of alfalfa mosaic virus (AMV RNA 4 5′ leader) tandemly stacked with at least one cellular translational enhancer element, and (b) an operably linked polynucleotide encoding a polypeptide of interest. The 36-base pair AMV RNA4 5′ leader is set forth in SEQ ID NO: 3. As for other translational enhancer elements, polynucleotide constructs of the invention may comprise functional variants and fragments of the AMV RNA4 5′ leader, as defined elsewhere herein.

In other embodiments, the polynucleotide constructs of the invention comprise (a) at least one copy of the translation leader sequence from the maize necrotic streak virus (MNeSV 5′ leader) tandemly stacked with at least one cellular translational enhancer element, and (b) an operably linked polynucleotide encoding a polypeptide of interest. The 122-base pair MNeSV 5′ leader is set forth in SEQ ID NO: 19. As for other translational enhancer elements, polynucleotide constructs of the invention may comprise functional variants and fragments of the MNeSV 5′ leader, as defined elsewhere herein.

In other embodiments, the polynucleotide constructs of the invention comprise (a) at least one viral translational enhancer element tandemly stacked with at least one translational enhancer element from an alcohol deydrogenase gene, and (b) an operably linked polynucleotide encoding a polypeptide of interest. Translational enhancer elements from alcohol dehydrogenase genes are known in the art, and include, but are not limited to, the 84-bp translation leader sequence for the tobacco ADH gene (NtADH 5′ leader), as set forth in SEQ ID NO:4, the translation leader sequence for the rice ADH2 gene (OsADH2 5′ leader, as set forth in SEQ ID NO: 5, the translation leader sequence for the Arabidopsis thaliana ADH gene (AtADH 5′ leader), as set forth in SEQ ID NO: 6, the translation leader sequence for the maize ADH gene (ADH 5′ leader), as set forth in SEQ ID NO:7, or functional variants or fragments thereof.

In still other embodiments, the polynucleotide constructs of the invention comprise (a) at least one viral translational enhancer element tandemly stacked with at least one translational enhancer element from a heat shock protein (HSP) gene, and (b) an operably linked polynucleotide encoding a polypeptide of interest. Translational enhancer elements from heat shock protein genes are known in the art, and include, but are not limited to, the translation leader sequence for the maize HSP101 gene (HSP101 5′ leader), as set forth in SEQ ID NO: 8, and the translation leader sequences for the maize HSP70, petunia HSP70, and soybean HSP17.9 genes, as set forth in SEQ ID NOs: 9, 10, and 11, respectively (see, e.g., U.S. Pat. Nos. 5,659,122 and 5,362,865, herein incorporated by reference in their entirety), or functional variants or fragments thereof.

Thus, in some embodiments, the present invention provides polynucleotide constructs comprising at least one translational enhancer element selected from the TMV, TEV, AMV RNA4, and MNeSV 5′ leaders set forth in SEQ ID NOs: 1, 2 (or 18), 3, and 19, respectively, tandemly stacked with at least one translational enhancer element selected from the group consisting of the tobacco, rice, Arabidopsis, and maize ADH 5′ leaders set forth in SEQ ID NOs:4, 5, 6, and 7, respectively; and (b) an operably linked polynucleotide encoding a polypeptide of interest. In some of these embodiments, the polynucleotide constructs of the invention comprise the TMV Ω 5′ leader set forth in SEQ ID NO: 1 (or a functional fragment or variant thereof as defined herein below) tandemly stacked with the tobacco ADH 5′ leader set forth in SEQ ID NO: 4 (or a functional fragment or variant thereof as defined herein below), and (b) an operably linked polynucleotide encoding a polypeptide of interest.

In other embodiments, the present invention provides polynucleotide constructs comprising at least one translational enhancer element selected from the TMV, TEV, AMV, and MNeSV 5′ leaders set forth in SEQ ID NOs: 1, 2 (or 18), 3, and 19, respectively, tandemly stacked with at least one translational enhancer element selected from the group consisting of the 5′ leaders for the maize HSP101 gene, maize HSP70 gene, petunia HSP70 gene, and soybean HSP17.9 gene, as set forth in SEQ ID NOs: 8, 9, 10, and 11, respectively; and (b) an operably linked polynucleotide encoding a polypeptide of interest. In some of these embodiments, the polynucleotide constructs of the invention comprise the TMV Ω 5′ leader set forth in SEQ ID NO: 1 (or a functional fragment or variant thereof as defined herein below) tandemly stacked with the maize HSP101 5′ leader set forth in SEQ ID NO: 8 (or a functional fragment or variant thereof as defined herein below), and (b) an operably linked polynucleotide encoding a polypeptide of interest.

As noted above, functional fragments and variants of a viral or cellular 5′ UTR can be utilized as a translational enhancer element within the polynucleotide constructs of the invention. As used herein, “functional” means that a fragment or variant nucleic acid sequence is capable of providing for increased expression of a polypeptide of interest when tandemly stacked with the other translational enhancer element (which may also be a fragment or variant of a full-length 5′ UTR) within a polynucleotide construct of the invention. As used herein, “fragment” means any portion of the 5′ UTR of interest. Fragments of a 5′ UTR may range from at least about 10 contiguous nucleotides up to the number of nucleotides present in the full-length 5′ UTR. Thus, in some embodiments, a fragment of a 5′ UTR is at least about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250 contiguous nucleotides in length, or any such value in between about 5 contiguous nucleotides and up to one less nucleotide than the full-length 5′ UTR.

Thus, for example, where the viral translational enhancer element is the TMV 5′ UTR (Ω; see SEQ ID NO: 1), TEV 5′ UTR (SEQ ID NO: 2 or SEQ ID NO: 18), AMV 5′ UTR (SEQ ID NO: 3), or MNeSV 5′ UTR, a fragment of this sequence may be tandemly linked to a cellular translational enhancer element so long as it is functional, i.e., is capable of providing for increased expression of a polypeptide of interest when tandemly stacked with at least one cellular translational enhancer element within a polynucleotide construct of the invention. For Ω, such a fragment may comprise at least about 5, 10, 15, 20, 25, 30, 35, 40, 45, 50, 55, 60, 65, or up to one less nucleotide than is present in the full-length Ω sequence (i.e., up to 67 contiguous nucleotides out of the 68 nucleotides set forth in SEQ ID NO: 1). For the TEV 5′ UTR, such a fragment may comprise at least about 5, 10, 15, 20, 25, 30, 35, 40, 45, 50, 55, 60, 65, 70, 75, 80, 85, 90, 95, 100, 105, 110, 115, 120, 125, 130, 135, 140, or up to one less nucleotide than is present in the full-length TEV 5′ UTR sequence (i.e., up to 142 contiguous nucleotides out of the 143 nucleotides set forth in SEQ ID NO: 2; or up to 143 contiguous nucleotides out of the 144 nucleotides set forth in SEQ ID NO: 18). For the AMV 5′ UTR, such a fragment may comprise at least about 5, 10, 15, 20, 25, 30, or up to one less nucleotide than is present in the full-length TEV 5′ UTR sequence (i.e., up to 35 contiguous nucleotides out of the 36 nucleotides set forth in SEQ ID NO: 3). For the MNeSV 5′ UTR, such a fragment may comprise at least about 5, 10, 15, 20, 25, 30, 35, 40, 45, 50, 55, 60, 65, 70, 75, 80, 85, 90, 95, 100, 105, 110, 115, 120, or up to one less nucleotide than is present in the full-length MNeSV 5′ UTR sequence (i.e., up to 121 contiguous nucleotides out of the 122 nucleotides set forth in SEQ ID NO: 19).

Similarly, where the cellular translational enhancer element is, for example, the tobacco ADH 5′ UTR (see SEQ ID NO: 4), the rice ADH2 5′ UTR (see SEQ ID NO: 5), the Arabidopsis ADH 5′ UTR (see SEQ ID NO: 6), the maize ADH 5′ UTR (see SEQ ID NO: 7), the maize heat shock protein 101 (HSP101) 5′ UTR (see SEQ ID NO:8), the maize heat shock protein 70 (HSP70) 5′ UTR (see SEQ ID NO: 9), the petunia heat shock protein 70 (HSP70) 5′ UTR (see SEQ ID NO: 10), or the soybean heat shock protein 17.9 (HSP17.9) 5′ UTR (see SEQ ID NO: 11), a fragment of this sequence may be tandemly linked to a viral translational enhancer element so long as it is functional, i.e., is capable of providing for increased expression of a polypeptide of interest when tandemly stacked with at least one viral translational enhancer element within a polynucleotide construct of the invention. For the tobacco ADH 5′ UTR, such a fragment may comprise at least about 5, 10, 15, 20, 25, 30, 35, 40, 45, 50, 55, 60, 65, 70, 75, 80, or up to one less nucleotide than is present in the full-length tobacco ADH 5′ UTR sequence (i.e., up to 83 contiguous nucleotides out of the 84 nucleotides set forth in SEQ ID NO: 4). For the rice ADH2 5′ UTR, such a fragment may comprise at least about 5, 10, 15, 20, 25, 30, 35, 40, 45, 50, 55, 60, 65, 70, 75, 80, 85, 90, 95, or up to one less nucleotide than is present in the full-length rice ADH2 5′ UTR sequence (i.e., up to 100 contiguous nucleotides out of the 101 nucleotides set forth in SEQ ID NO: 5). For the Arabidopsis ADH 5′ UTR, such a fragment may comprise at least about 5, 10, 15, 20, 25, 30, 35, 40, 45, 50, 55, or up to one less nucleotide than is present in the full-length Arabidopsis ADH 5′ UTR sequence (i.e., up to 57 contiguous nucleotides out of the 58 nucleotides set forth in SEQ ID NO:6). For the maize ADH 5′ UTR, such a fragment may comprise at least about 5, 10, 15, 20, 25, 30, 35, 40, 45, 50, 55, 60, 65, 70, 75, 80, 85, 90, 95, 100, 105, or up to one less nucleotide than is present in the full-length maize ADH 5′ UTR sequence (i.e., up to 106 contiguous nucleotides out of the 107 nucleotides set forth in SEQ ID NO: 7). For the maize HSP101 5′ UTR, such a fragment may comprise at least about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or up to one less nucleotide than is present in the full-length maize HSP101 5′ UTR sequence (i.e., up to 205 contiguous nucleotides out of the 206 nucleotides set forth in SEQ ID NO:8). For the maize HSP70 5′ UTR, such a fragment may comprise at least about 5, 10, 15, 20, 25, 30, 35, 40, 45, 50, 55, 60, 65, 70, 75, 80, 85, 90, 95, 100, 105, or up to one less nucleotide than is present in the full-length maize HSP70 5′ UTR sequence (i.e., up to 106 contiguous nucleotides out of the 107 nucleotides set forth in SEQ ID NO: 9). For the petunia HSP70 5′ UTR, such a fragment may comprise at least about 5, 10, 15, 20, 25, 30, 35, 40, 45, 50, 55, 60, 65, 70, 75, 80, 85, 90, or up to one less nucleotide than is present in the full-length petunia HSP70 5′ UTR sequence (i.e., up to 95 contiguous nucleotides out of the 96 nucleotides set forth in SEQ ID NO: 10). For the soybean HSP17.9 5′ UTR, such a fragment may comprise at least about 5, 10, 15, 20, 25, 30, 35, 40, 45, 50, 55, 60, 65, 70, or up to one less nucleotide than is present in the full-length soybean HSP17.9 5′ UTR sequence (i.e., up to 71 contiguous nucleotides out of the 72 nucleotides set forth in SEQ ID NO: 11).

As used herein, “variants” of a 5′ UTR means sequences having substantial similarity with the nucleotide sequence for that 5′ UTR (e.g., the 5′ UTR set forth in SEQ ID NO: 1, 2, 3, 4, 5, 6, 7, 8, 9, 10, 11, 18, or 19) or with a fragment thereof. For 5′ UTR, naturally occurring variants such as these can be identified with the use of well-known molecular biology techniques, as, for example, with PCR and hybridization techniques. Variant nucleotide sequences also include synthetically derived nucleotide sequences, such as those generated, for example, by using site-directed mutagenesis, the techniques of which are also well known to those of skill in the art. See, e.g., Sambrook et al., Molecular Cloning: A Laboratory Manual (Cold Spring Harbor Laboratory Press, Plainview, N.Y. 2001) and Current Protocols in Molecular Biology (Ausubel et al. eds., Greene Publishing and Wiley-Interscience, New York 1995); herein incorporated by reference in their entirety.

Generally, variants of a particular 5′ UTR, including variants of any of SEQ ID NOs: 1-11, 18, and 19, will have at least about 40%, 50%, 60%, 65%, 70%, generally at least about 75%, 80%, 85%, preferably at least about 90%, 91%, 92%, 93%, 94%, 95%, 96%, 97%, 98% or 99% sequence identity to that particular nucleotide sequence as determined by sequence alignment programs described herein below using default parameters. Variants of a 5′ UTR of interest will be functional, that is a variant is capable of providing for increased expression of a polypeptide of interest when tandemly stacked with the other translational enhancer element (which may also be a fragment or variant of a full-length 5′ UTR) within a polynucleotide construct of the invention. Biologically active variants include, for example, the native or naturally occurring 5′ UTR having one or more nucleotide substitutions, deletions, or insertions.

As used herein, “sequence identity” or “identity” in the context of two nucleic acid sequences makes reference to the nucleotides in the two sequences that are the same when aligned for maximum correspondence over a specified comparison window. As used herein, “comparison window” means a contiguous and specified segment of a polynucleotide sequence, where the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 nucleotides, or longer.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., San Diego, Calif.). Alignments using these programs can be performed using the default parameters.

Assembly of the polynucleotide constructs of the invention, and their insertion in an expression cassette of interest, can be carried out using genetic engineering techniques well known to those of skill in the art. See, e.g., Sambrook et al. (2001), supra; and Ausubel et al., supra. Furthermore, means for preparing recombinant vectors that are suitable for introducing a polynucleotide construct into a plant are well known in the art. As used herein, “vector,” “construct,” or “vector construct” means any recombinant polynucleotide construct that may be used for the purpose of plant transformation, i.e., the introduction of a heterologous polynucleotide into a host plant cell. See, e.g., the vectors described in U.S. Pat. Nos. 4,971,908, 4,940,835, 4,769,061, and 4,757,011; Rodriguez, Vectors: A Survey of Molecular Cloning Vectors and Their Uses (Butterworths, Boston 1988); Glick et al., Methods in Plant Molecular Biology and Biotechnology (CRC Press 1993); and Ausubel et al. (1995), supra; and Sambrook et al. (2001), supra). Typical constructs useful for introduction of nucleic acids into plants are well known in the art and include vectors derived from the tumor-inducing (Ti) plasmid of Agrobacterium tumefaciens (Rogers et al. (1987) Meth. Enzymol. 153:253-277).

The polynucleotide constructs of the invention can be utilized in transient assays to assess the effects of the tandemly stacked translational enhancer elements on expression of a polypeptide of interest. For example, mRNAs comprising the tandemly stacked viral and cellular translational enhancer elements operably linked to the mRNA transcript encoding the polypeptide of interest can be constructed using standard methods known to those of skill in the art, for example, by in vitro transcription of the corresponding cDNA, and the resultant mRNAs introduced into a plant cell of interest, where translation of the mRNA can be monitored. See, e.g., the transient assays described in Gallie et al. (1987) Nucleic Acids Res. 15:8693-8711, and Jobling and Gehrke (1987) Nature 325:622-625.

In other embodiments, the polynucleotide constructs of the invention are incorporated within an expression cassette and introduced into plant of interest for transient or stable in vivo transcription and translation of the encoded polypeptide of interest.

### Expression Cassettes

The polynucleotide constructs of the invention can be operably linked to regulatory elements that provide for expression of the operably linked polynucleotide encoding a polypeptide of interest. In this manner, the present invention provides expression cassettes and expression vectors comprising a polynucleotide construct of the invention, which comprises (a) at least one viral translational enhancer element tandemly stacked with at least one cellular translational enhancer element, and (b) an operably linked polynucleotide encoding a polypeptide of interest. “Expression cassette” as used herein means a nucleic acid molecule capable of directing expression of a polynucleotide sequence of interest in an appropriate host cell, and thus comprises 5′ and 3′ regulatory sequences operably linked to the polynucleotide sequence of interest (i.e., a polynucleotide construct of the invention). The operably linked elements within the expression cassette are configured so that there is a functional linkage between them, and thus each element within the expression cassette is capable of carrying out its intended function. The operably linked elements may be contiguous or non-contiguous.

The expression cassettes of the present invention comprise a polynucleotide construct of the invention, and thus are chimeric constructs, i.e., the nucleic acid sequence for at least one of their components is heterologous (i.e., foreign or not naturally occurring together) with respect to the nucleic acid sequence for at least one of their other components. Thus, for example, the tandemly stacked viral and cellular translational enhancer elements are heterologous to each other as they are derived from a different source (i.e., viral versus cellular). In like manner, the regulatory region, for example, a promoter, may be heterologous to the coding sequence for the polypeptide of interest. It is also recognized that one or more of the individual components within an expression cassette of the invention may be native to another component within the expression cassette. For example, the promoter may be the naturally occurring promoter for the encoded polypeptide of interest, although the two components are not found in their native configuration.

The expression cassettes of the present invention are heterologous to the plant cell into which they are introduced, i.e., the particular DNA sequence of the expression cassette does not occur naturally in the host plant cell and must have been introduced into the host plant cell or an ancestor of the host plant cell by a transformation event. In any case, any one or more of the individual components within the expression cassette may be native to the plant host (i.e., the nucleotide sequence for the component itself can be found as a naturally occurring sequence within the genome of that plant host) or may be heterologous to the plant host (i.e., the nucleotide sequence for the component itself is foreign to the plant host, either by virtue of being from a different organism or by virtue of genetic modification of its original form, for example, by nucleotide substitution, insertion, deletion, and/or truncation).

Exemplary regulatory sequences for use in an expression cassette of the invention include, but are not limited to, promoter sequences, polyadenylation signals, transcription termination sequences, upstream regulatory domains, origins of replication, internal ribosome entry sites (“IRES”), other translational enhancers (e.g., 3′ UTRs), and the like, which collectively provide for replication and transcription of an operably linked polynucleotide of interest, and translation of any coding sequence therein, in a recipient plant cell of interest. Not all of these control sequences need always be present so long as the polynucleotide of interest is capable of being replicated, transcribed, and translated in the recipient plant cell. Regulatory sequences therefore can be a regulatory region of DNA usually comprising a TATA box capable of directing RNA polymerase II to initiate RNA synthesis at the appropriate transcription initiation site for a particular coding sequence. An expression control sequence can additionally comprise other recognition sequences generally positioned upstream or 5′ to the TATA box, which influence (e.g., enhance) the transcription initiation rate. Furthermore, an expression control sequence may additionally comprise sequences generally positioned downstream or 3′ to the TATA box, which influence the transcription initiation rate.

An expression cassette of the invention typically comprises in the 5′-3′ direction of transcription a promoter that is functional in a plant, an operably linked polynucleotide construct of the invention, and an operably linked translational termination region that is functional in a plant. In some embodiments, the expression cassette comprises a selectable marker gene to allow for selection of stable transformants. Alternatively, a selectable marker may be provided in an additional expression cassette, on the same vector, or on different vectors. The expression of the polynucleotide construct in the expression cassette may be under the control of a constitutive promoter or of an inducible promoter that initiates transcription only when the host cell is exposed to some particular external stimulus. Additionally, the promoter can also be specific to a particular tissue or organ or stage of development. The cassette may also contain at least one additional polynucleotide of interest (e.g., a coding sequence for another polypeptide of interest) to be cotransformed into the plant of interest. Alternatively, the additional polynucleotide(s) of interest can be provided on multiple expression cassettes, on the same vector or on different vectors. The expression cassettes of the invention are provided with a plurality of restriction sites and/or recombination sites for insertion of a polynucleotide construct of the invention that is to be under the transcriptional regulation of the regulatory regions.

Any promoter that is functional in a plant cell (i.e., is capable of driving expression of an operably linked transcribable polynucleotide in a plant cell) may be operably linked to a polynucleotide construct of the invention. The promoter may be the native (i.e., naturally occurring) promoter for the coding region of the polynucleotide construct, or may be a promoter that is heterologous (i.e., foreign or not naturally occurring) to the coding region of the polynucleotide construct. Where the promoter is not the naturally occurring promoter for the coding region of the polynucleotide construct, it may be heterologous due to genetic manipulation of the naturally occurring promoter sequence and/or naturally occurring coding sequence (e.g., by substitution, insertion, deletion, and/or truncation of nucleotides within the naturally occurring promoter and/or coding sequence), or may be heterologous due to its genetic source of origin (e.g., a promoter from another gene and/or another organism). In some embodiments, the promoter is the native promoter for the coding region of the polynucleotide construct, and both sequences are native to the plant host into which the expression cassette is introduced (i.e., the promoter and the coding sequence are derived from the same gene that is naturally found within the plant host). Alternatively, the promoter is the native promoter for the coding region of the polynucleotide construct, but both sequences are heterologous (i.e., foreign or not naturally occurring) to the plant host into which the expression cassette is introduced (i.e., the promoter and the coding sequence of the polynucleotide construct are both foreign to the plant host, for example, by virtue of genetic manipulation of their original sequence or by virtue of being from another genetic source). In yet other embodiments, the promoter is heterologous to the coding sequence, and is either native to the plant host (i.e., the promoter is derived from a gene of the plant host), or is foreign to the plant host (i.e., its original sequence has been manipulated, or it is from another genetic source). The foregoing relationships (i.e., heterologous versus native) between the promoter, coding sequence, and plant host into which the expression cassette of the invention is introduced are not intended to be limiting, but are merely exemplary in nature.

The choice of promoters to be included in an expression cassette of the invention depends upon several factors, including, but not limited to, efficiency, selectability, inducibility, desired expression level of the polypeptide of interest, and cell- or tissue-preferential expression of the polypeptide. It is a routine matter for one of skill in the art to modulate the expression of an operably linked polynucleotide sequence by appropriately selecting and positioning promoters and other regulatory regions relative to that sequence. Methods for identifying and characterizing promoter regions in plant genomic DNA include, e.g., those described in Jordano et al. (1989) Plant Cell 1:855-866; Bustos et al. (1989) Plant Cell 1:839-854; Green et al. (1988) EMBO J. 7:4035-4044; Meier et al. (1991) Plant Cell 3:309-316; and Zhang et al. (1996) Plant Physiology 110:1069-1079.

The promoters that are used for expression of a polynucleotide construct of the invention can be a strong plant promoter, a viral promoter, or a chimeric promoter composed of elements such as: TATA box from any gene (or synthetic, based on analysis of plant gene TATA boxes), optionally fused to the region 5′ to the TATA box of plant promoters (which direct tissue and temporally appropriate gene expression), optionally fused to 1 or more enhancers (such as the Cauliflower Mosaic Virus (CaMV) 35S enhancer, FMV enhancer, CMP enhancer, RUBISCO small subunit enhancer, plastocyanin enhancer (see, e.g., Chua et al. (2003) Plant Cell 15(6):1468-1479), and activating elements derived from the Agrobacterium tumefaciens octopine synthase gene (see, U.S. Pat. No. 5,955,646).

Alternatively, a weak plant promoter can be used to alter the effects of gene silencing in a plant host cell of interest. Thus, for example, where expression of a target polypeptide of interest within a host plant cell has been inhibited by, for example, a gene suppression technique such as antisense or hairpin RNA interference, a polynucleotide construct of the invention comprising tandemly stacked translational enhancer elements operably linked to a coding sequence for the target polypeptide can be operably linked to a weak promoter and introduced into the plant host cell by any method known in the art to provide for low level expression of the target polypeptide.

In such embodiments of the invention, a “weak promoter” is a promoter that provides for low level expression of the operably linked coding sequence for the target polypeptide. However, it is recognized that weak promoters may be those that provide for expression of the operably linked coding sequence in only a few cells and not in others to give a total low level of expression of the target polypeptide within the plant host. Weak plant promoters may be naturally occurring or may represent variants of a naturally occurring promoter sequence that have been modified to decrease the level of expression of an operably linked coding sequence for the target polypeptide, or truncated versions of a naturally occurring promoter sequence that provide for decreased expression of the target polypeptide. Examples of weak promoters include, for example, the core promoter of the Rsyn7 promoter (WO 99/43838 and U.S. Pat. No. 6,072,050), the core 35S CaMV promoter, and the like.

In some embodiments, constitutive expression of a polynucleotide construct of the invention is desirable. Constitutive promoters provide for unregulated, and thus continuous, expression of the operably linked polynucleotide construct. Exemplary constitutive promoters include, for example, the core promoter of the Rsyn7 promoter and other constitutive promoters disclosed in WO 99/43838 and U.S. Pat. No. 6,072,050; the core CaMV ³⁵S promoter (Odell et al. (1985) Nature 313:810-812); rice actin promoter (McElroy et al. (1990) Plant Cell 2:163-171); ubiquitin promoter (Christensen et al. (1989) Plant Mol. Biol. 12:619-632; and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, e.g., those disclosed in U.S. Pat. Nos. 5,608,149; 5,608,144; 5,604,121; 5,569,597; 5,466,785; 5,399,680; 5,268,463; 5,608,142; and 6,177,611; 7,256,276; 7,550,578; the disclosures of which are herein incorporated by reference in their entirety.

Appropriate plant or chimeric promoters are useful for applications such as expression of a polynucleotide construct of the invention in certain tissues, while minimizing expression in other tissues, such as seeds, or reproductive tissues. Exemplary cell type- or tissue-preferential promoters drive expression preferentially in the target tissue, but may also lead to some expression in other cell types or tissues as well. Thus, promoters can be chosen that give tissue-specific expression (e.g., root, leaf and floral-specific promoters). See, e.g., the promoters described in Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505. See also, the promoters disclosed in U.S. Pat. Nos. 7,297,839 and 7,129,397, which provide for preferential expression in plastids.

Promoters active in photosynthetic tissue in order to drive transcription in green tissues such as leaves and stems are also encompassed by the present invention. Most suitable are promoters that drive expression only or predominantly in such tissues. The promoter may confer such expression constitutively throughout the green tissues, or differentially with respect to the green tissues, or differentially with respect to the developmental stage of the green tissue in which expression occurs, or in response to external stimuli.

Examples of such promoters include the ribulose-1,5-bisphosphate carboxylase (RbcS) promoters such as the RbcS promoter from eastern larch (Larix laricina), the pine cab6 promoter (Yamamoto et al. (1994) Plant Cell Physiol. 35:773-778), the Cab-1 gene promoter from wheat (Fejes et al. (1990) Plant Mol. Biol. 15:921-932), the CAB-1 promoter from spinach (Lubberstedt et al. (1994) Plant Physiol. 104:997-1006), the cab1R promoter from rice (Luan et al. (1992) Plant Cell 4:971-981), the pyruvate orthophosphate dikinase (PPDK) promoter from corn (Matsuoka et al. (1993) Proc. Natl. Acad. Sci. USA 90:9586-9590), the tobacco Lhcb1*2 promoter (Cerdan et al. (1997) Plant Mol. Biol. 33:245-255), the Arabidopsis thaliana SUC2 sucrose-H+ symporter promoter (Truernit et al. (1995) Planta 196:564-570), and thylakoid membrane protein promoters from spinach (psaD, psaF, psaE, PC, FNR, atpC, atpD, cab, rbcS. Other promoters that drive transcription in stems, leafs and green tissue are described in U.S. Patent Publication No. 2007/0006346, herein incorporated by reference in its entirety. Likewise, a maize gene encoding phosphoenol carboxylase (PEPC) has been described in the art (Hudspeth and Grula (1989) Plant Mol. Biol. 12: 579-589). Using standard molecular biological techniques, the promoter for this gene can be used to drive the expression of a polynucleotide construct of the invention in a green tissue-specific manner in plants.

In other embodiments of the present invention, inducible promoters may be desired. Inducible promoters drive transcription in response to external stimuli such as chemical agents or environmental stimuli. For example, inducible promoters can confer transcription in response to environmental stimuli (e.g., heat shock gene promoters, drought-inducible gene promoters, pathogen-inducible gene promoters, wound-inducible gene promoters, and light/dark-inducible gene promoters), or plant growth regulators (e.g., promoters from genes induced by abscissic acid, auxins, cytokinins and gibberellic acid). See, e.g., U.S. Pat. Nos. 7,199,286 and 7,230,159.

Other promoters that can be used to drive expression of a polynucleotide construct of the invention include, but are not limited to, the Cauliflower Mosaic Virus 35S promoter, opine synthetase promoters (e.g., nos, mas, ocs, etc.), ubiquitin promoter, actin promoter, ribulose bisphosphate (RubP) carboxylase small subunit promoter, alcohol dehydrogenase promoter, developmental promoters (see, e.g., U.S. Pat. Nos. 6,953,848 and 6,437,221), and the chimeric promoter described in U.S. Pat. No. 6,987,179. The RubP carboxylase small subunit promoter is known in the art (Silverthorne et al. (1990) Plant Mol. Biol. 15:49-58). Other promoters from viruses that infect plants also are suitable including, but not limited to, promoters isolated from Dasheen mosaic virus, Chlorella virus (e.g., the Chlorella virus adenine methyltransferase promoter; Mitra and Higgins (1994) Plant Mol. Biol. 26:85-93), tomato spotted wilt virus, tobacco rattle virus, tobacco necrosis virus, tobacco ring spot virus, tomato ring spot virus, cucumber mosaic virus, peanut stump virus, alfalfa mosaic virus, sugarcane baciliform badnavirus, and the like.

A variety of transcriptional terminators are available for use in expression cassettes comprising a polynucleotide construct of the invention. These are responsible for the termination of transcription beyond the coding region of the polynucleotide contruct within the expression cassette and correct mRNA polyadenylation. The termination region may be native (i.e., naturally occurring) with the promoter, may be native with the operably linked coding sequence within the polynucleotide construct, may be native with the plant into which the expression cassette is to be introduced, or may be derived from another source (i.e., foreign or heterologous to the promoter, the coding sequence, or the plant), or any combination thereof. Appropriate transcriptional terminators are those that are known to function in plants and include, for example, the CAMV 35S terminator, the tml terminator, the nopaline synthase terminator, and the pea rbcs E9 terminator.

In some embodiments, the expression cassette will comprise a selectable marker gene for the selection of transformed cells. In other embodiments, the selectable marker gene is constructed within another expression cassette, on the same vector or a different vector, and the polynucleotide construct of the invention and the selectable marker are cotransformed into the plant or plant part of interest. Selectable markers used routinely in transformation include the nptll gene, which confers resistance to kanamycin and related antibiotics (Messing and Vierra (1982) Gene 19:259-268; Bevan et al. (1983) Nature 304:184-187); the bar gene, which confers resistance to the herbicide phosphinothricin (White et al. (1990) Nucleic Acids Res. 18:1062; Spencer et al. (1990) Theor. Appl. Genet. 79:625-631); the hph gene, which confers resistance to the antibiotic hygromycin (Blochinger and Diggelmann (1984) Mol. Cell. Biol. 4:2929-2931); the dhfr gene, which confers resistance to methatrexate (Bourouis et al. (1983) EMBO J. 2:1099-1104); the EPSPS gene, which confers resistance to glyphosate (U.S. Pat. Nos. 4,940,935 and 5,188,642); and the phosphomannose isomerase gene (PMI), which provides the ability to metabolize mannose (U.S. Pat. Nos. 5,767,378 and 5,994,629). Other suitable selectable markers are known in the art, and any such marker can be utilized in the practice of the present invention.

Furthermore, where desirable, the expression cassette can be designed to target the expressed polypeptide of interest to a particular organelle of the plant cell (e.g., to the mitochondria or a plastid such as a chloroplast), or target the polypeptide for extracellular secretion. Various mechanisms for targeting gene products are known to exist in plants and the sequences controlling the functioning of these mechanisms have been characterized in some detail.

For example, the targeting of gene products to the chloroplast is controlled by a transit peptide found at the amino terminal end of various proteins, which is cleaved during chloroplast import to yield the mature protein (Comai et al. (1988) J. Biol. Chem. 263:15104-15109). These transit peptides can be fused to heterologous polypeptide products to effect the import of these products into the chloroplast (van den Broeck et al. (1985) Nature 313:358-363). DNA encoding for appropriate transit peptides can be isolated from the 5′ end of the cDNAs encoding the RUBISCO protein, the CAB protein, the EPSP synthase enzyme, the GS2 protein, and many other proteins that are known to be chloroplast localized. See also, the section entitled “Expression with Chloroplast Targeting” in Example 37 of U.S. Pat. No. 5,639,949; herein incorporated by reference in its entirety.

The above-described mechanisms for cellular targeting can be utilized not only in conjunction with their native promoters, but also in conjunction with heterologous promoters so as to effect a specific cell-targeting goal under the transcriptional regulation of a promoter that has an expression pattern different from that of the promoter from which the targeting transit peptide derives.

Thus, where a polynucleotide construct of the invention encodes a polypeptide that is to be targeted to the chloroplast, the coding sequence within the construct may be a fusion polynucleotide comprising sequence encoding an appropriate chloroplast-targeting transit peptide fused in frame to a sequence encoding the polypeptide of interest. In order to ensure localization in the plastids, it is conceivable to use, for example, the transit peptide sequence for plastidic Ferredoxin: NADP+ oxidoreductase (FNR) of spinach, which is disclosed in Jansen et al. (1988) Current Genetics 13:517-522. Another example is the transit peptide sequence of the waxy protein of maize including the first 34 amino acid residues of the mature waxy protein (Klosgen et al. (1989) Mol. Gen. Genet. 217:155-161). It is also possible to use this transit peptide without the first 34 amino acids of the mature protein. Furthermore, the transit peptide sequences of the ribulose bisposphate carboxylase small subunit (Wolter et al. (1988) Proc. Natl. Acad. Sci. USA 85:846-850; Nawrath et al. (1994) Proc. Natl. Acad. Sci. USA 91:12760-12764), of NADP malate dehydrogenase (Galiardo et al. (1995) Planta 197:324-332), of glutathione reductase (Creissen et al. (1995) Plant J. 8:167-175), or of the R1 protein (Lorberth et al. (1998) Nature Biotechnology 16:473-477) can be used.

Where it is desirable for the polypeptide of interest to be secreted, for example, into the cell wall or into a culture medium, a polynucleotide construct of the invention can be designed such that the coding sequence within the construct is a fusion polynucleotide comprising sequence encoding an appropriate signal peptide fused in frame to a sequence encoding the polypeptide of interest. As used herein, “signal peptide” or “signal sequence” means a nucleic acid sequence that encodes a polypeptide that interacts with a receptor protein on the membrane of the endoplasmic reticulum (ER) to direct the transport of a growing polypeptide chain across the membrane and into the ER for secretion from the cell. This signal peptide is often cleaved from the precursor polypeptide to produce a “mature” polypeptide lacking the signal peptide. As such, the polynucleotide constructs within an expression cassette can be designed such that the encoded polypeptide is secreted into the cell wall or secreted from the plant, e.g., into a culture medium.

The polynucleotide constructs of the invention can use any suitable signal sequence known in the art (including bacterial, yeast, fungal, insect, mammalian, and plant signal sequences). See, e.g., U.S. Pat. No. 6,020,169. The signal peptide can correspond to a signal peptide of the polypeptide of interest. Suitable signal peptides are well known to those of skill in the art.

The expression cassettes described herein can comprise other regulatory sequences that have been found to enhance gene expression from within an expression cassette in order to increase the expression of the polynucleotide construct contained therein. For example, various intron sequences have been shown to enhance expression, particularly in monocotyledonous cells. Intron 1 was found to be particularly effective and enhanced expression in fusion constructs with the chloramphenicol acetyltransferase gene (Callis et al. (1987) Genes Develop. 1:1183-1200). See also, U.S. Pat. No. 6,342,660 describing the use of the maize alcohol dehydrogenase intron. Intron sequences have been routinely incorporated into plant transformation vectors, typically within a non-translated leader. Thus, the expression cassettes comprising a polynucleotide construct of the invention may further comprise an operably linked intron sequence therein. In some embodiments, an intron sequence is inserted within one or more of the tandemly stacked viral and cellular translational enhancer elements.

It is recognized that there are known differences between the optimal translation initiation context nucleotide sequences for translation initiation codons in different organisms, and the composition of these translation initiation context nucleotide sequences can influence the efficiency of translation initiation. See, e.g., Lukaszewicz et al. (2000) Plant Science 154:89-98; and Joshi et al. (1997) Plant Mol. Biol. 35:993-1001. As used herein, “translation initiation codon” means the codon that initiates translation of the coding region within an mRNA transcribed from a nucleic acid molecule of interest. The translation initiation codon is usually ATG in the DNA sequence, and AUG in the mRNA transcript. As used herein, “translation initiation context nucleotide sequence” means an identity of three nucleotides directly 5′ of the translation initiation codon. As such, the translation initiation context nucleotide sequence for the translation initiation codon of a coding sequence within a polynucleotide construct of the invention may be modified to enhance expression in plants by selecting a plant-preferred translation initiation context nucleotide sequence. Thus, the polynucleotide constructs of the invention can use any suitable translation initiation context nucleotide sequence known in the art, especially one from a plant. For example, the polynucleotide construct of the invention can be modified such that the three nucleotides directly upstream of the translation initiation codon of the coding sequence within the polynucleotide construct of interest are “ACC,” “ACA,” or “AAAAAA.”

Furthermore, any coding sequence contained within an expression cassette described herein can be optimized for expression in the plant into which it is to be introduced. That is, the nucleotide sequences can be synthesized using plant-preferred codons for improved expression, or may be synthesized using codons at a plant-preferred codon usage frequency. Generally, the GC content of the gene will be increased. See, e.g., Campbell and Gown (1990) Plant Physiol. 92:1-11 for a discussion of host-preferred codon usage. Methods are available in the art for synthesizing plant-preferred genes. See, e.g., U.S. Pat. Nos. 5,380,831, and 5,436,391, and Murray et al. (1989) Nucleic Acids Res. 17:477-498, herein incorporated by reference. Tables showing the frequency of codon usage based on the sequences contained in GenBank® releases may be found on the website for the Kazusa DNA Research Institute in Chiba, Japan. This database is described in Nakamura et al. (2000) Nucleic Acids Res. 28:292.

After constructing an expression cassette described herein, it is introduced into a plant of interest by any suitable transformation method known in the art, including those described herein below.

### Polypeptides of Interest

The tandemly stacked viral and cellular translational enhancer elements within the polynucleotide constructs of the invention can be used to enhance expression of any polypeptide of interest. In this manner, the operably linked coding sequence within a polynucleotide construct of the invention can encode polypeptides that are useful for genetic manipulation of metabolic pathways to improve agronomic performance of plants, e.g., increased disease resistance, herbicide resistance, nutrient utilization, and environmental stress resistance; to alter agronomic characteristics, e.g., modifications in starch, oil, fatty acid, or protein content/composition to enhance animal and human nutrition, improve digestibility, and/or improve processing traits; and to develop modifications, such as male sterility, senescence, and the like; and to introduce transgene expression of pharmaceuticals, industrial enzymes, and the like.

Thus, the polynucleotide constructs of the invention can comprise a coding sequence for a polypeptide that provides a desirable characteristic associated with plant morphology, physiology, growth and development, yield, nutritional enhancement, disease or pest resistance, or environmental or chemical tolerance. The expression of such polypeptides is desirable in order to confer an agronomically important trait. Examples of polypeptides that provide a beneficial agronomic trait to crop plants may be, e.g., polypeptides conferring insect control (U.S. Pat. Nos. 7,244,820; 7,230,167; 6,809,078; 6,780,408; 6,720,488; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,342,660; 6,326,351; 6,320,100; 6,313,378; 6,300,544; 6,284,949; 6,281,413; 6,281,016; 6,278,041; 6,277,823; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; and 5,763,241); fungal disease resistance (U.S. Pat. Nos. 7,098,378; 6,864,076; 6,864,068; 6,653,280; 6,573,361; 6,506,962; 6,316,407; 6,300,103; 6,215,048; 5,516,671; 5,773,696; 6,121,436; 6,316,407; and 6,291,647); virus resistance (U.S. Pat. Nos. 6,617,496; 6,608,241; 6,015,940; 6,013,864; 5,850,023; and 5,304,730); nematode resistance (U.S. Pat. Nos. 6,784,337 and 6,228,992); bacterial disease resistance (U.S. Pat. Nos. 7,098,378; 6,956,115; 6,528,702; and 5,516,671); herbicide resistance (U.S. Pat. Nos. 7,312,379; 7,056,715; 6,803,501; 6,448,476; 6,307,129; 6,294,345; 6,248,876; 6,225,114; 6,107,549; 5,866,775; 5,804,425; 5,633,435; and 5,463,175; and U.S. Patent Application Publication Nos. 2003/0135879 and 2003/0115626); plant growth and development (U.S. Pat. Nos. 6,723,897; 6,603,064; and 6,518,488); starch production (U.S. Pat. Nos. 6,538,181; 6,538,179; 6,538,178; 5,750,876; and 6,476,295); increased yield (U.S. Pat. RE38,446; U.S. Pat. Nos. 6,716,474; 6,663,906; 6,476,295; 6,441,277; 6,423,828; 6,399,330; 6,372,211; 6,235,971; 6,222,098; and 5,716,837); modified oils production (U.S. Pat. Nos. 6,444,876; 6,426,447; and 6,380,462); high oil production (U.S. Pat. Nos. 6,495,739; 5,608,149; 6,483,008; and 6,476,295); modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018); high protein production (U.S. Pat. No. 6,380,466); fruit ripening (U.S. Pat. No. 5,512,466); enhanced animal and human nutrition (U.S. Pat. Nos. 6,723,837; 6,653,530; 6,5412,59; 5,985,605; and 6,171,640); biopolymers (U.S. Pat. No. RE37,543; U.S. Pat. Nos. 6,228,623; 5,958,745; and U.S. Patent Application Publication No. 2003/0028917); environmental stress resistance (U.S. Pat. No. 6,072,103); pharmaceutical peptides and secretable peptides (U.S. Pat. Nos. 6,812,379; 6,774,283; and 6,140,075; 6,080,560); improved processing traits (U.S. Pat. No. 6,476,295); improved digestibility (U.S. Pat. No. 6,531,648); low raffinose (U.S. Pat. No. 6,166,292); industrial enzyme production (U.S. Pat. No. 5,543,576); improved flavor (U.S. Pat. No. 6,011,199), nitrogen fixation (U.S. Pat. No. 5,229,114); hybrid seed production (U.S. Pat. No. 5,689,041); fiber production (U.S. Pat. Nos. 6,576,818; 6,271,443; 5,981,834; and 5,869,720); and biofuel production (U.S. Pat. No. 5,998,700); the contents of each of these patents and patent application publications is herein incorporated by reference in their entirety.

As noted above, and where applicable, the polypeptide of interest may be expressed as part of a fusion polypeptide.

The polynucleotide constructs of the invention comprising the tandemly stacked viral and cellular translational enhancer elements can thus comprise a polynucleotide encoding any polypeptide of interest. These constructs can be introduced into any plant of interest in order to improve agronomic performance, alter agronomic characteristics, and provide for expression of pharmaceuticals, industrial enzymes, and the like.

### Plants of Interest

The invention thus provides transformed (i.e., transgenic) plants and plant parts thereof comprising a polynucleotide construct of the invention, wherein the construct comprises: a) at least one viral translational enhancer element tandemly stacked with at least one cellular translational enhancer element; and b) a polynucleotide encoding a polypeptide of interest. As used herein, “plant part” means plant organs (e.g., leaves, stems, roots, etc.), seeds, and plant cells. Plant parts also include, without limitation, protoplasts, tissues, nodules, callus, plant cell tissue cultures from which plants can be regenerated, embryos, as well as flowers, ovules, anthers, pollen, stems, branches, fruits, kernels, ears, cobs, husks, stalks, leaves, tillers, roots, root tips, and the like originating in plants or their progeny. Plant cells also include, without limitation, cells of seeds, embryos, meristematic regions, callus tissue, leaves, roots, shoots, gametophytes, sporophytes, pollen, and microspores.

As used herein, “transformed” or “transgenic” means a plant or plant part thereof into which has been introduced a foreign polynucleotide molecule, such as a polynucleotide construct of the invention. The introduced polynucleotide molecule may be integrated into the genomic DNA of the recipient plant or plant part such that the introduced polynucleotide molecule is inherited by subsequent progeny. A “transgenic” or “transformed” plant or plant part thereof, for example, a cell or a tissue, also includes progeny of the plant or plant part, and progeny produced from a breeding program employing such a transgenic plant or plant part as a parent in a cross and exhibiting an altered phenotype resulting from the presence of a foreign polynucleotide molecule, e.g., a polynucleotide construct of the invention.

In preferred embodiments, the polynucleotide construct of the invention and an operably linked promoter that functions within a plant cell are stably integrated within the genome of the plant or plant part thereof so that the desired characteristic or trait provided by the polypeptide encoded thereby can be capable of being inherited by the progeny thereof, more particularly, by the progeny of multiple successive generations. In some embodiments, the polynucleotide construct of the invention is stably integrated into the genome of the plant or plant part thereof as part of an expression cassette of the invention, and thus the plant or plant part thereof has been genetically modified by way of introduction of this expression cassette into one or more cells of the plant or plant part thereof.

Plants according to the present invention include any plant that is cultivated for the purpose of producing plant material that is sought after by man or animal for either oral consumption, or for utilization in an industrial, pharmaceutical, or commercial process. The invention may be applied to any of a variety of plants, including, but not limited to maize, wheat, rice, barley, soybean, cotton, sorghum, beans in general, rape/canola, alfalfa, flax, sunflower, safflower, millet, rye, sugarcane, sugar beet, cocoa, tea, Brassica, cotton, tobacco, coffee, sweet potato, flax, peanut, clover; vegetables such as lettuce, tomato, cucurbits, cassaya, potato, carrot, radish, pea, lentils, cabbage, cauliflower, broccoli, Brussels sprouts, peppers, and pineapple; tree fruits such as citrus, apples, pears, peaches, apricots, walnuts, avocado, banana, and coconut; and flowers such as orchids, carnations and roses. Other plants useful in the practice of the invention include perennial grasses, such as switchgrass, prairie grasses, Indian grass, Big bluestem grass, and the like.

### Methods of the Invention

The polynucleotide constructs of the invention find use in methods for increasing expression of a polypeptide of interest in a plant or plant part thereof. The methods of the invention comprise introducing into a plant or plant part thereof a polynucleotide construct of the invention operably linked to a promoter that is functional in a plant. When cultured under conditions suitable for expression of a polynucleotide construct of the invention, the tandemly stacked translational enhancer elements provide for greater efficiency in translation of the related mRNA transcript. The methods of the present invention thus provide for increased expression of a polypeptide of interest in a plant or plant part thereof. As used herein, “expression” means the synthesis of the encoded polypeptide, including the transcription, translation, and assembly of the encoded polypeptide. Increased expression of the polypeptide is in the context of a comparison between any two plants or plant parts, e.g., expression of the polypeptide in a plant or plant part that has been genetically modified by way of introduction of a polynucleotide construct of the invention, versus the expression of that polypeptide in a corresponding wild-type plant or wild-type plant part.

In some embodiments, the increased expression is in the context of a comparison between a plant or plant part that has been genetically modified by way of introduction of a polynucleotide construct of the invention, versus the expression of that polypeptide in a corresponding control plant or control plant part. As used herein, “control plant” or “control plant part” means a plant or plant part that has been genetically modified to express the same polypeptide from a polynucleotide construct that differs from the polynucleotide construct of the invention only in the absence of the tandemly stacked viral and cellular translational enhancer elements. In this manner, the control plant or plant part comprises a polynucleotide construct that contains the same transcriptional regulatory region (i.e., the same promoter), the same coding sequence for the polypeptide, and either of the following: no operably linked translational enhancer elements, or only a single operably linked translational enhancer element, with that element being either the same viral translational enhancer element as is present in the polynucleotide construct of the invention, or the same cellular translational enhancer element as is present in the polynucleotide construct of the invention.

In particular embodiments of the invention, the level of expression of the polypeptide of interest is increased in a transgenic plant or plant part of the invention by at least about 10%, 15%, 20%, 25%, 30%, 35%, 40%, 45%, 50%, 55%, 50%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 225%, 250%, 275%, 300%, 350%, 400%, 450%, or at least about 500% when compared to a wild-type plant or plant part, or to a control plant or plant part. In other embodiments of the invention, the level of expression of the polypeptide of interest is increased in a transgenic plant or plant part of the invention by at least about 1-fold, 1.5-fold, 2-fold, 2.5-fold, 3-fold, 3.5-fold, 4-fold, 4.5-fold, or at least about 5-fold when compared to a wild-type plant or plant part, or to a control plant or plant part. The expression level of the polypeptide of interest may be measured directly, for example, by assaying for the level of the polypeptide expressed in the plant or plant part, for example, by measuring the activity of the polypeptide in the plant or plant part.

The polynucleotide constructs and expression cassettes of the invention can be introduced into a plant or plant part of interest using any plant transformation techniques known to those of skill in the art, including, but not limited to electroporation (as illustrated in U.S. Pat. No. 5,384,253); microprojectile bombardment (as illustrated in U.S. Pat. Nos. 6,403,865; 5,015,580; 5,550,318; 5,538,880; 6,160,208; 6,399,861; and 6,403,865); Agrobacterium-mediated transformation (as illustrated in U.S. Pat. Nos. 7,029,908; 5,824,877; 5,591,616; 5,981,840; and 6,384,301); and protoplast transformation (as illustrated in U.S. Pat. No. 5,508,184); all of which are incorporated herein by reference. These constructs and expression cassettes may also be introduced into a plant or plant part of interest using a breeding protocol. The following description of plant transformation techniques is provided for guidance and is not intended to be limiting.

### Plant Transformation and Breeding.

The polynucleotide constructs and expression cassettes of the invention, alone or in combination with one or more additional nucleic acid molecules of interest, are transformed into a cell of a target plant of interest. These constructs and expression cassettes can be introduced into the plant cell in a number of art-recognized ways. As used herein, “introducing” in the context of a polynucleotide, means a polynucleotide construct or expression cassette of the invention is presented to the plant in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the plant cell of interest in a single transformation event, in separate transformation events, or, e.g., as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

As used herein, “transient transformation” or “transient expression” in the context of a polynucleotide, means that a polynucleotide is introduced into the plant and does not integrate into the genome of the plant. Transient transformation and transient expression can be achieved using any suitable method known in the art. For example, transient expression can be performed with plant cell cultures or by infiltrating plant leaves with recombinant Agrobacterium strains. Transient expression is not inherited by the progeny of the plant.

As used herein, “stably introducing” or “stably introduced,” in the context of a polynucleotide introduced into a plant, means that the introduced polynucleotide is stably incorporated into the plant genome, and thus the plant is stably transformed with the polynucleotide. “Stable transformation” or “stably transformed” means that a polynucleotide, e.g., a polynucleotide construct or expression cassette of the invention, introduced into a plant integrates into the genome of the plant and is capable of being inherited by the progeny thereof, more particularly, by the progeny of multiple successive generations.

Numerous transformation vectors available for plant transformation are known to those of ordinary skill in the plant transformation arts, and the polynucleotide constructs and expression cassettes of the invention can be used in conjunction with any such vectors. The selection of vector will depend upon the preferred transformation technique and the target plant species for transformation. For certain target species, different antibiotic or herbicide selectable markers may be preferred. Selectable markers used routinely in transformation include those selectable markers described herein above.

Methods and vectors for transforming plants are well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Many vectors are available for transformation using Agrobacterium tumefaciens. These typically carry at least one T-DNA border sequence and include vectors such as pBIN19 (Bevan (1984) Nucleic Acids Res. 12:8711-8721). For the construction of vectors useful in Agrobacterium transformation, see, e.g., U.S. Patent Application Publication No. 2006/0260011 and U.S. Pat. No. 7,029,908, herein incorporated by reference in their entirety.

Transformation without the use of Agrobacterium tumefaciens circumvents the requirement for T-DNA sequences in the chosen transformation vector and consequently vectors lacking these sequences can be utilized in addition to vectors such as the ones described above, which contain T-DNA sequences. Transformation techniques that do not rely on Agrobacterium include transformation via particle bombardment, protoplast uptake (for example, PEG and electroporation), and microinjection. The choice of vector depends largely on the preferred selection for the plant species being transformed. For the construction of such vectors, see, e.g., U.S. Patent Application Publication No. 2006/0260011, herein incorporated by reference.

Where it is desirable to introduce a polynucleotide construct or expression cassette of the invention into plant plastids, plastid transformation vector pPH143 (WO 97/32011, see, Example 36) is used. The expression cassette is inserted into pPH143 thereby replacing the PROTOX coding sequence. This vector is then used for plastid transformation and selection of transformants for spectinomycin resistance. Alternatively, the expression cassette is inserted in pPH143 so that it replaces the aadH gene. In this case, transformants are selected for resistance to PROTOX inhibitors. See also, the plastid transformation techniques disclosed in U.S. Pat. No. 7,235,711, herein incorporated by reference in its entirety.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al. (1984) EMBO J. 3:2717-2722; Potrykus et al. (1985) Mol. Gen. Genet. 199:169-177; Reich et al. (1986) Biotechnology 4:1001-1004; and Klein et al. (1987) Nature 327:70-73. In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g., pCIB200 or pCIB2001) to an appropriate Agrobacterium strain, which may depend on the complement of vir genes carried by the host Agrobacterium strain either on a co-resident Ti plasmid or chromosomally (e.g., strain CIB542 for pCIB200 and pCIB2001 (Uknes et al. (1993) Plant Cell 5:159-169). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen and Willmitzer (1988) Nucl. Acids Res. 16: 9877).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a polynucleotide of interest involves propelling inert or biologically active particles at plant tissues and cells. This technique is disclosed in U.S. Pat. Nos. 4,945,050, 5,036,006, and 5,100,792, herein incorporated by reference in their entirety. Generally, this procedure involves propelling inert or biologically active particles at the cells under conditions effective to penetrate the outer surface of the cell and afford incorporation within the interior thereof. When inert particles are utilized, the vector can be introduced into the plant cell by coating the particles with the vector containing the desired gene. Alternatively, the target cell can be surrounded by the vector so that the vector is carried into the cell by the wake of the particle. Biologically active particles (e.g., dried yeast cells, dried bacterium, or a bacteriophage, each containing DNA sought to be introduced) can also be propelled into plant cell tissue. Transformation of most monocotyledonous species has now also become routine. Preferred techniques include direct gene transfer into protoplasts using PEG or electroporation techniques, and particle bombardment into callus tissue. Transformations can be undertaken with a single DNA species or multiple DNA species (i.e., co-transformation) and both of these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. (1986) Biotechnology 4:1093-1096).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) published techniques for transformation of A188-derived maize line using particle bombardment. Furthermore, WO 93/07278 and Koziel et al. (Biotechnology 11:194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment. See also, the biolistic transformation methods disclosed in U.S. Pat. No. 6,403,865, herein incorporated by reference in its entirety.

Transformation of rice can also be undertaken by direct gene transfer techniques utilizing protoplasts or particle bombardment. Protoplast-mediated transformation has been described for Japonica-types and Indica-types (Zhang et al. (1988) Plant Cell Rep 7: 379-384; Shimamoto et al. (1989) Nature 338:274-277; and Datta et al. (1990) Biotechnology 8:736-740). Both types are also routinely transformable using particle bombardment (Christou et al. (1991) Biotechnology 9:957-962). Furthermore, WO 93/21335 describes techniques for the transformation of rice via electroporation.

Patent Application EP 0 332 581 describes techniques for the generation, transformation, and regeneration of Pooideae protoplasts. These techniques allow the transformation of Dactylis and wheat. Furthermore, wheat transformation has been described by Vasil et al. (Biotechnology 10:667-674 (1992)) using particle bombardment into cells of type C long-term regenerable callus, and also by Vasil et al. (Biotechnology 11:1553-1558 (1993)) and Weeks et al. (Plant Physiol. 102:1077-1084 (1993)) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashige and Skoog (1962) Physiologia Plantarum 15: 473-497) and 3 mg/L 2,4-D for induction of somatic embryos, which is allowed to proceed in the dark. On the chosen day of bombardment, embryos are removed from the induction medium and placed onto the osmoticum (i.e., induction medium with sucrose or maltose added at the desired concentration, typically 15%). The embryos are allowed to plasmolyze for 2-3 hours and are then bombarded. Twenty embryos per target plate are typical, although not critical. An appropriate gene-carrying plasmid (such as pCIB3064 or pSOG35) is precipitated onto micrometer size gold particles using standard procedures. Each plate of embryos is shot with the DuPont BIOLISTICS® helium device using a burst pressure of about 1000 psi using a standard 80 mesh screen. After bombardment, the embryos are placed back into the dark to recover for about 24 hours (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/L NAA, 5 mg/L GA), further containing the appropriate selection agent (10 mg/L basta in the case of pCIB3064 and 2 mg/L methotrexate in the case of pSOG35). After approximately one month, developed shoots are transferred to larger sterile containers known as “GA7s,” which contain half-strength MS, 2% sucrose, and the same concentration of selection agent.

Transformation of monocotyledons using Agrobacterium has also been described. See, WO 94/00977 and U.S. Pat. No. 5,591,616, both of which are incorporated herein by reference; see also, Negrotto et al. (2000) Plant Cell Reports 19:798-803, incorporated herein by reference.

For example, rice (Oryza sativa) can be used for generating transgenic plants. Various rice cultivars can be used (Hiei et al. (1994) Plant Journal 6:271-282; Dong et al. (1996) Molecular Breeding 2:267-276; and Hiei et al. (1997) Plant Molecular Biology 35:205-218). Also, the various media constituents described below may be either varied in quantity or substituted. Embryogenic responses are initiated and/or cultures are established from mature embryos by culturing on MS-CIM medium (MS basal salts, 4.3 g/liter; B5 vitamins (200×), 5 ml/L; Sucrose, 30 g/L; proline, 500 mg/L; glutamine, 500 mg/Lr; casein hydrolysate, 300 mg/L; 2,4-D (1 mg/ml), 2 ml/L; adjust pH to 5.8 with 1 N KOH; Phytagel, 3 g/L). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/L spectinomycin and any other appropriate antibiotic) for about 2 days at 28° C. Agrobacterium is re-suspended in liquid MS-CIM medium. The Agrobacterium culture is diluted to an optical density (OD) at 600 of 0.2-0.3 and acetosyringone is added to a final concentration of 200 μM. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/L) to inhibit the growth of Agrobacterium. For constructs utilizing the PMI selectable marker gene (Reed et al. (2001) In Vitro Cell. Dev. Biol.-Plant 37:127-132), cultures are transferred to selection medium containing Mannose as a carbohydrate source (MS with 2% Mannose, 300 mg/L Ticarcillin) after 7 days, and cultured for 3-4 weeks in the dark. Resistant colonies are then transferred to regeneration induction medium (MS with no 2,4-D, 0.5 mg/liter IAA, 1 mg/L zeatin, 200 mg/L timentin 2% Mannose and 3% Sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% Sorbitol) for 2 weeks and then moved to the greenhouse when they are large enough and have adequate roots. Plants are transplanted to soil in the greenhouse (T₀ generation) grown to maturity, and the T₁ seed is harvested.

The cells that have been transformed with a polynucleotide construct or expression cassette of the present invention may be grown into plants in accordance with conventional ways. See, e.g., McCormick et al. (1986) Plant Cell Reports 5:81-84. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide construct or expression cassette of the invention stably incorporated into their genome.

The plants obtained via transformation with a polynucleotide construct or expression cassette of the present invention can be any of a wide variety of plant species, including those of monocots and dicots; as well as the list of agronomically important crops set forth elsewhere herein. The polynucleotide constructs and expression cassettes of the invention in combination with other characteristics important for production and quality can be incorporated into plant lines through breeding. Breeding approaches and techniques are known in the art. See, e.g., Welsh, Fundamentals of Plant Genetics and Breeding (John Wiley and Sons, NY 1981); Crop Breeding (Wood ed., American Society of Agronomy Madison, Wis. 1983); Mayo, The Theory of Plant Breeding (2″ ed.; Clarendon Press, Oxford 1987); Singh, Breeding for Resistance to Diseases and Insect Pests (Springer-Verlag, NY 1986); and Wricke and Weber, Quantitative Genetics and Selection Plant Breeding (Walter de Gruyter and Co., Berlin 1986).

The genetic properties engineered into the transgenic seeds and plants described above are passed on by sexual reproduction or vegetative growth and can thus be maintained and propagated in progeny plants. Generally, maintenance and propagation make use of known agricultural methods developed to fit specific purposes such as tilling, sowing, or harvesting.

### Detection of Stable Integration and Expression of the Polypeptide of Interest.

The above conditions lead to regeneration of green plantlets and plants with photosynthetic ability. As described above, the test used for confirmation that the nucleic acid molecule of interest is stably integrated into the genome of the plant of interest, or a plant part thereof, necessarily depends on the property to be conferred to the plant. For example, when the property is herbicide resistance, confirmation may be achieved by treatment of the growing plants by spraying or painting the leaves with the herbicide in a concentration that is lethal for control plants that have not been subjected to the transformation process.

Expression of the polypeptide of interest in the transformed plant or part thereof may be detected using an immunological method. Immunological methods that can be used include, but are not limited to, competitive and non-competitive assay systems using immune-based techniques such as Western blots, radioimmunoassays, ELISA (enzyme linked immunosorbent assay), multiplex ELISA, “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and the like. Such assays are routine and known in the art (see, e.g., Ausubel et al. (1994), supra).

In addition to immunoassays, expression can be measured by evaluating patterns of expression of the polynucleotide encoding the polypeptide of interest, or of reporter genes, or both. For example, expression patterns can be evaluated by Northern analysis, PCR, RT-PCR, Taq Man analysis, ribonuclease protection assays, FRET detection, monitoring one or more molecular beacons, hybridization to an oligonucleotide array, hybridization to a cDNA array, hybridization to a polynucleotide array, hybridization to a liquid microarray, hybridization to a microelectric array, cDNA sequencing, clone hybridization, cDNA fragment fingerprinting, and the like. The particular method elected will be dependent on such factors as quantity of RNA recovered, artisan preference, available reagents and equipment, detectors, and the like.

The following examples are offered by way of illustration and not by way of limitation.

### EXPERIMENTAL

### Example 1

### Vectors

Twenty vectors were constructed to test the dual enhancer concept. Two sets of nine vectors were generated for tobacco or corn expression. Each enhancer and enhancer combination was tested in both corn and tobacco. Two additional vectors were tested only in tobacco. The Cestrum promoter and 35S terminator were used for tobacco expression and the PEPC promoter and PEPC terminator were used for corn expression. Endoglucanase targeted to the ER was used as the reporter gene. The endoglucanase gene was codon optimized for the expression host: corn optimization for corn expression and soy optimization for tobacco expression. Care was taken to keep the context between the promoter and enhancer and between the enhancer and the initiation codon (Kozak) the same for all constructs in corn or tobacco. In cases where double enhancers were used, the sequences were contiguous with no intervening sequence between the enhancers. Tobacco: Promoter—TGCGGATCC—Enhancer Insertion—AAAAAA—Reporter Corn: Promoter—GGATCC—Enhancer Insertion—TAAACC—Reporter. Vector construction is set forth in more detail below.

### Example 2

### Tandemly Stacked Translational Enhancer Elements Enhance Expression in a Tobacco Transient System

The results generated in the tobacco transient system shows that endoglucanase (EG) expression was significantly increased in tobacco plant leaves transformed with a polynucleotide construct in which two translational enhancer elements (viral+ cellular 5′ UTRs) were positioned in tandem relative to one another when compared to control constructs. Enhanced expression was observed when the viral translational enhancer element was positioned upstream (i.e., at the 5′ end) of the cellular translational enhancer element.

### Methods

Plant material: A transient expression assay using TEV-B tobacco transformants was used to monitor expression level of EG provided by the various polynucleotide constructs.

Polynucleotide Constructs: Six different polynucleotide constructs were used. The constructs contained various combinations of viral and cellular 5′ UTRs positioned upstream of a sequence encoding EG. The constructs were used for the tobacco transient and stable systems.

1. Tandem constructs:

Ω-NtADH construct: In this construct, the viral translational enhancer element (5′ UTR (5′ leader) of the Tobacco Mosaic Virus (TMV), also indicated as “Ω” (SEQ ID NO: 1) was tandemly stacked with the cellular translational enhancer element 5′ UTR of the tobacco alcohol dehydrogenase (NtADH) gene (SEQ ID NO: 4). The elements of this construct were positioned in the following 5′-3′ order: (1) a Cestrum promoter from yellow leaf curling virus (SEQ ID NO: 12; (2) the Ω enhancer (SEQ ID NO: 1); (3) the NtADH translational enhancer element (SEQ ID NO: 4); (4) a 6 bp Soy Kozak sequence (SEQ ID NO: 13); (5) a signal sequence from soybean glycinin seed protein (SEQ ID NO:14); (6) an endoglucanase-encoding sequence (SEQ ID NO: 15); (7) an ER retention signal (SEQ ID NO:16); and (8) a t35s transcription terminator (SEQ ID NO: 17).

NtADH-Ω construct: In this construct, the cellular translational enhancer element (5′ UTR of the NtADH gene (SEQ ID NO:4) was tandemly stacked with the viral translational enhancer element (5′ UTR of the TMV (SEQ ID NO:1). The elements of this construct were positioned in the following 5′-3′ order: (1) a Cestrum promoter from yellow leaf curling virus (SEQ ID NO:12); (2) the NtADH enhancer element (SEQ ID NO:4); (3) the Ω enhancer element (SEQ ID NO:1); (4) a 6 bp Soy Kozak sequence (SEQ ID NO:13); (5) a signal sequence from soybean glycinin seed protein (SEQ ID NO:14); (6) an endoglucanase-encoding sequence (SEQ ID NO:15); (7) an ER retention signal (SEQ ID NO:16); and (8) the t35s transcription terminator (SEQ ID NO:17).

2. Single Constructs:

Ω construct: The elements of this construct were positioned in the following 5′-3′ order: (1) a Cestrum promoter from yellow leaf curling virus (SEQ ID NO:12); (2) the enhancer element (SEQ ID NO:1); (3) a 6 bp Soy Kozak sequence (SEQ ID NO:13); (4) a signal sequence from soybean glycinin seed protein (SEQ ID NO:14); (5) an endoglucanase-encoding sequence (SEQ ID NO:15); (6) an ER retention signal (SEQ ID NO:16); and (7) the t35s transcription terminator (SEQ ID NO:17).

NtADH construct: The elements of this construct were positioned in the following 5′-3′ order: (1) a Cestrum promoter from yellow leaf curling virus (SEQ ID NO:12); (2) the NtADH translational enhancer element (SEQ ID NO: 4); (3) a 6 bp Soy Kozak sequence (SEQ ID NO: 13); (4) a signal sequence from soybean glycinin seed protein (SEQ ID NO: 14); (5) an endoglucanase-encoding sequence (SEQ ID NO:15); (6) an ER retention signal (SEQ ID NO: 16); and (7) the t35s transcription terminator (SEQ ID NO: 17).

Transient Transformation Protocol: Expression cassettes were cloned into a binary vector. The binary vector was transferred into Agrobacterium tumefaciens strain LBA4404 using the freeze-thaw method (An et al. (1988) “Binary vector,” A3 1-19, in Plant Molecular Biology Manual, ed. Gelvin and Schilproot (Kluwar Academic Publishers, Dordrecht).

Leaves from young TEV-B plants (4 weeks old) were used for transient expression of enzymes. Transgenic TEV-B tobacco plants (made in the tobacco cultivar Xanthi) containing a mutated P1/HC-Pro gene from TEV that suppresses post-transcriptional gene silencing (Mallory et al. (2002) Nat. Biotechnol. 20:622-625) were used for transient expression of selected enzymes in tobacco leaves. Preparation of Agrobacterium cultures and infiltration of tobacco was carried out as described by Azhakanandam et al. (2007) Plant Mol. Biol. 63:393-404. Briefly, genetically modified Agrobacteria were grown overnight in 50 ml of LB medium containing 100 μM acetosyringone and 10 μM MES (pH 5.6), and subsequently were pelleted by centrifugation at 4000×g for 10 min. The pellets were resuspended in the infection medium (Murashige and Skoog salts with vitamins, 2% sucrose, 500 μM MES (pH 5.6), 10 μM MgSO₄, and 100 μM acetosyringone) to OD₆₀₀=1.0 and subsequently held at 28° C. for 3 hours. Infiltration of individual leaves was carried out on about 4 week old TEV-B tobacco plants using a 5 ml syringe by pressing the tip of the syringe (without a needle) against the abaxial surface of the leaf. Infiltrated plants were maintained at 22-25° C. with a photoperiod of 16 hours light and 8 hours dark. Plant tissue was harvested after 5 days post infiltration for subsequent analysis.

Activity Assay: The method measured EG in nmol/min/mg of protein in terms of liberated glucose produced on CM-cellulose at 40° C., pH 4.75. Glucose oxidase/peroxidase (GOPOD) chemistry was used to measure glucose relative to a standard curve. The glucose-based assay method is a colorimetric assay in which GOPOD reacts with glucose at 40° C. to generate a light to dark pinkish chromophore. The assay consists of four (4) basic steps: (1) grinding/milling transgenic tissue; (2) weighing out ground tissue samples; (3) extracting enzyme in Na-acetate buffer; and (4) assaying for enzymatic activity/protein quantification.

1. Materials:

Sodium-acetate buffer solution: 100 mM Na-acetate (pH 4.75), 0.02% NaN₃, 0.02% Tween, and 1 complete protease inhibitor cocktail tablet per 50 ml of buffer. The buffer can be prepared and stored up to 3 months at 4° C.; following addition of the cocktail tablet, the buffer solution has a shelf-life of one week at 4° C. The solution was prepared by mixing 50 ml of Na-acetate (1M), 50 ml acetic acid (1M) in about 800 ml of H₂O, with pH adjusted to 4.75. Then, 10 ml of Na-azide (2%) and 10 ml Tween (2%) were added and diluted to 1 L with H₂O.

Substrate solution: A 0.5% carboxymethyl cellulose (CMC-4M; Megazyme Lot #81101; Wicklow, Ireland) solution was prepared by weighing 5 g of CMC-4M into a dry 1000 ml volumetric flask. The sample was wet thoroughly with 25 ml of 95% ethanol and stirred while 600 ml of H₂O was added thereto. The solution was heated to 100° C. and stirred for 10 minutes to ensure that the substrate dissolved. The solution was then allowed to cool to 25° C., and then 50 ml of Na-acetate (1M), 50 ml of acetic acid (1M), and 10 ml of Na-azide (2%) were added and the volume adjusted to 1000 ml with H₂O. This substrate solution was stored at 4° C.

β-glucosidase solution: 20 μl of β-glucosidase from Aspergillus niger (Megazyme) was mixed per 1 ml of substrate to a final concentration of 0.8 μl/ml. This solution was made fresh daily.

Glucose standards: Concentrated glucose (100 mM) was prepared in Na-acetate buffer from anhydrous glucose (99.5% purity). Dilutions were made in Na-acetate buffer to generate solutions at 0, 1, 2, 3, 4 and 5 mM glucose. In the GOPOD assay, 20 μl of each standard was added to generate a standard curve of 0, 1, 20, 40, 60, 80 and 100 nmol.

Glucose reagent buffer (concentrate: 1M potassium dihydrogen orthophosphate; 200 mM para-hydroxybenzoic acid; and 0.4% sodium azide.

Glucose determination reagent (per vial): >12,000 U glucose oxidase; >650 U peroxidase; and 0.4 mmol 4-aminoantipyrine.

Glucose standard: 1.0 mg/ml glucose; and 0.2% w/v benzoic acid. Chromogen reagent (GOPOD): 50.0 ml of the glucose reagent buffer was diluted to 1 L with distilled H₂O. The contents of one (1) vial of the glucose determination reagent was dissolved in the glucose reagent buffer. The resulting GOPPOD reagent is stable for up to 3 months at 2-5° C. when stored in a brown reagent bottle or >12 month when stored in the frozen state. When this reagent is freshly prepared it may be light yellow or light pink in color. It will develop a stronger pink color over 2-3 months at 4° C. The absorbance of this solution should be less than 0.05 when read against distilled water.

2. Sample Prep and Extraction:

Green leaf samples/24 well block format: four ball bearings were added to each well of a 24-well block that was kept on dry ice. For each sample, ˜0.5 g of green leaf was transferred to each well of the block, and the block was sealed with a rubber cover and placed at −80° C. for a minimum of 3 hours. On the day of an assay, samples were ground using a Kleco® Titer Plate/Micro Tube Grinding Mill (Kleco; Visalia, Calif.) for 2 minutes and then briefly centrifuged at 3000 rpm for 30 seconds. The rubber cover was removed from the block and 1-3 ml of Na-acetate buffer was added. The 24-well block was then sealed with a plate sealer twice, once in each direction, and vortexed. Samples were then extracted at room temperature for 20 minutes on benchtop rotators. The 24-well block was centrifuged at 3000 rpm for 5 minutes. The supernatant was transferred to an archive plate (i.e., a 96-well flat-bottom reading plate or 96-deep well block), leaving the bottom row empty for standards.

3. Assay: 96-well PCR plates in duplicate on ice were prepared. For the Time 120 (T120) plate, 50 μl of the β-glucosidase and substrate mixture were added to each well of the plate. Then, 20 μl of sample extract was added. The plate was sealed, vortexed, and briefly centrifuged (3000 rpm for 15 seconds). Next, the plate was placed in a PCR machine at 40° C. for 2 hours.

Following the 2-hour incubation, 200 μl of GOPOD was added to each well of a 96-well flat bottom reading plate, to which 20 μl of the T120 sample or glucose standard was added. The plate was sealed, vortexed, and briefly centrifuged (3000 rpm for 15 seconds). Next, the plate was placed on a 40° C. hot plate for 20 minutes, and then absorption at 510 nm (light path of 1 cm) was read.

For a time 0 (T0) control plate, 50 μl of the β-glucosidase and substrate mixture were added to each well of the plate. Then, 20 μl of sample extract was added. The plate was sealed, vortexed, and briefly centrifuged. Next, the plate was placed in a PCR machine at 90° C. for 10 minutes.

Following the 10-minute incubation, 200 μl of GOPOD was added to each well of a 96-well flat bottom plate, to which 20 μl of the T0 sample or glucose standard was added. The plate was sealed, vortexed, and briefly centrifuged. Next, the plate was placed at 40° C. for 20 minutes and absorption at 510 nm (light path of 1 cm) was read.

Total protein: Total protein was measured with a Thermo Scientific Pierce BCA Protein Assay Kit (Thermo Fisher Scientific Inc.; Rockford, Ill.) according to the manufacturer's instructions.

Formula for calculations: Enzymatic activity=μmol/min/mg protein=(nmol glucose T120 sample−nmol glucose T0 sample)×( 1/120 minutes)×(1/0.02 ml spiked in enzyme r×n)=(nmol/min/ml)/(mg/ml total protein)=nmol/min/mg protein.

### Results

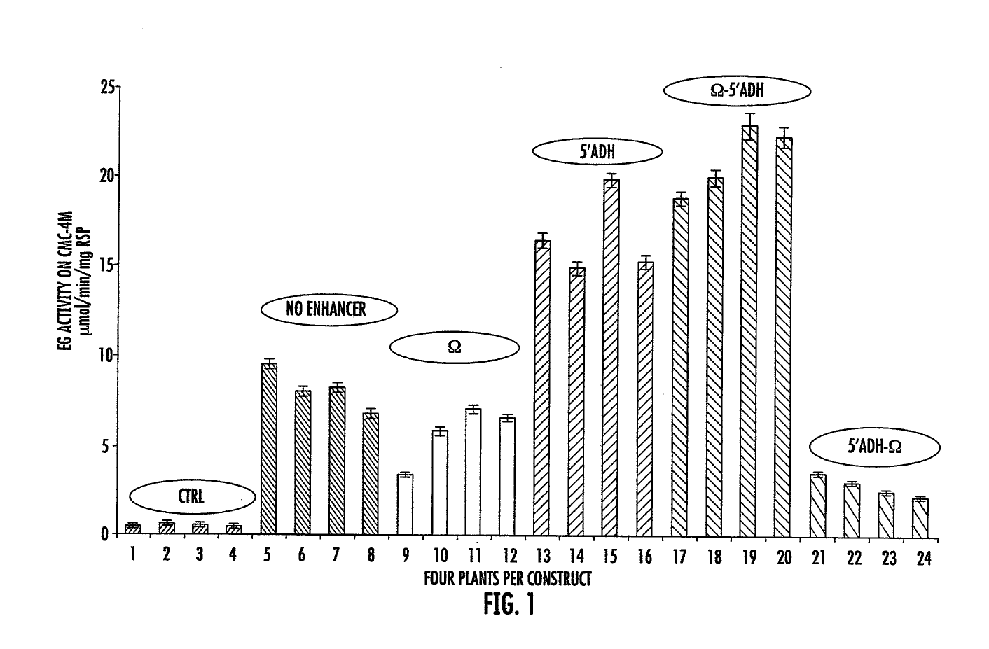

As shown in FIG. 1, the construct having no translational enhancer element present had endoglucanase (EG) activity that was at baseline (8 μmol/min/mg total soluble protein), and the vector control construct was below baseline. With respect to the single element constructs, the NtADH translational enhancer element increased activity about 1.5 times over baseline. In contrast, the Ω translational enhancer element had a negative effect on activity. With respect to the tandemly stacked translational enhancer element constructs, the order in which the cellular and viral translational enhancer elements were positioned significantly affected EG expression and ultimately EG activity. That is, when the Ω translational enhancer element was positioned upstream of the NtADH translational enhancer element, activity increased about 2.0 times over baseline. In contrast, when the NtADH translational enhancer element was positioned upstream of the Ω translational enhancer element, activity was below baseline. Endoglucanase expression therefore was affected by the tandem arrangement of translational enhancer elements. By positioning the viral translational enhancer element upstream of the cellular enhancer element, EG expression was enhanced by an additional ˜25% over that observed with the single cellular enhancer element.

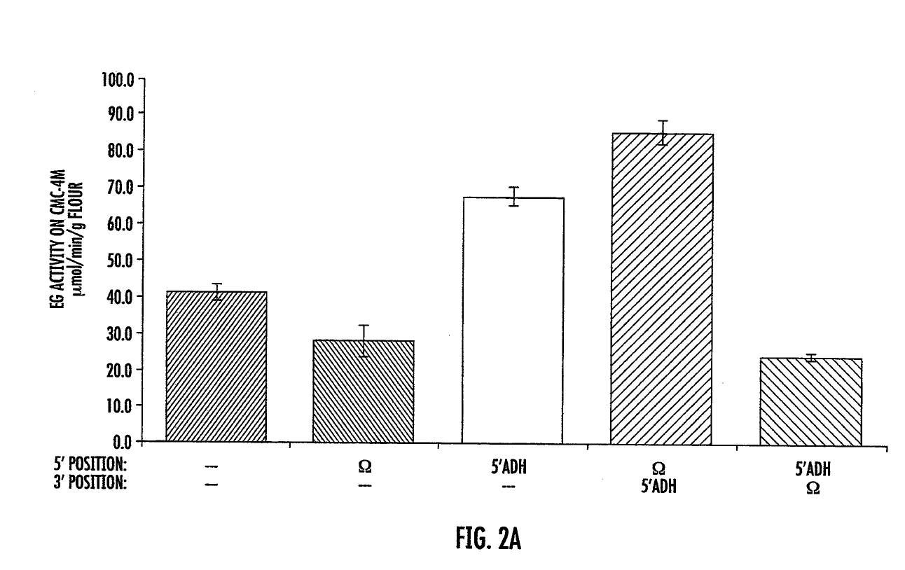

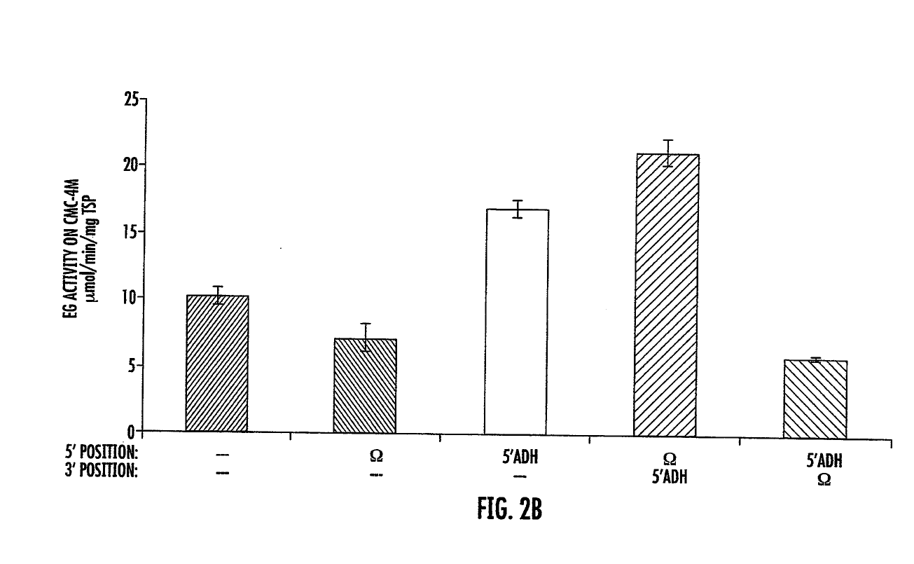

Because the order of the viral translational enhancer element relative to the cellular translational enhancer element appeared to influence expression and thus activity, a second set of experiments was performed using the five constructs described above (vector containing no translational enhancer elements; Ω construct; NtADH construct; Ω-NtADH construct; and NtADH-Ω construct. In a first experiment, expression from the five constructs was compared on a leaf fresh weight basis (FIG. 2A); in a second experiment, expression from the five constructs was compared on a total soluble protein basis (FIG. 2B). As above, the order of the viral and cellular translational enhancer elements significantly affected EG activity. Thus, EG activity increased above baseline with the NtADH translational enhancer element, and increased further only when the viral translational enhancer element was positioned upstream of the cellular translational enhancer element.

The degree to which the Ω-NtADH construct enhanced EG activity compared to the vector construct that had no enhancer elements present is summarized in Table 1.

TABLE 1
Enhanced Endoglucanse Activity in Ω-NtADH Constructs.

Endoglucanse Activity

| Experiment | No Enhancer | Ω-NtADH | Enhancement |
|---|---|---|---|
| 1 (AD003)*** | 40.9 | 85.2 | 2.1 fold |
| 2 (AD008) | 6.3 | 11.6 | 1.9 fold |
| 3 (AD010) | 8.1 | 21.0 | 2.6 fold |
| 4 (AD012) | 9.2 | 15.7 | 1.7 fold |

***AD003 activity was standardized to fresh leaf weight, not total soluble protein, and hence the higher activity for this experiment relative to the other three.

### Example 3

### Tandemly Stacked Translational Enhancer Elements Enhance Expression in Stable Tobacco Events

1. Tandem Constructs

Ω-NtADH construct: In this construct, the viral translational enhancer element 5′ UTR of the Tobacco Mosaic Virus (TMV), also indicated as “Ω” (SEQ ID NO:1) was tandemly stacked with the cellular translational enhancer element 5′ UTR of the tobacco alcohol dehydrogenase (NtADH) gene (SEQ ID NO:4). The elements of this construct were positioned in the following 5′-3′ order: (1) a Cestrum promoter from yellow leaf curling virus (SEQ ID NO:12; (2) the Ω enhancer (SEQ ID NO:1); (3) the NtADH translational enhancer element (SEQ ID NO: 4); (4) a 6 bp Soy Kozak sequence (SEQ ID NO: 13); (5) a signal sequence from soybean glycinin seed protein (SEQ ID NO: 14); (6) an endoglucanase-encoding sequence (SEQ ID NO: 15); (7) an ER retention signal (SEQ ID NO: 16); and (8) a t35s transcription terminator (SEQ ID NO: 17).

NtADH-Ω construct: In this construct, the cellular translational enhancer element (5′ UTR of the NtADH gene (SEQ ID NO: 4) was tandemly stacked with the viral translational enhancer element (5′ UTR of the TMV (SEQ ID NO: 1). The elements of this construct were positioned in the following 5′-3′ order: (1) a Cestrum promoter from yellow leaf curling virus (SEQ ID NO: 12); (2) the NtADH enhancer element (SEQ ID NO: 4); (3) the SZ enhancer element (SEQ ID NO: 1); (4) a 6 bp Soy Kozak sequence (SEQ ID NO: 13); (5) a signal sequence from soybean glycinin seed protein (SEQ ID NO: 14); (6) an endoglucanase-encoding sequence (SEQ ID NO: 15); (7) an ER retention signal (SEQ ID NO: 16); and (8) the t35s transcription terminator (SEQ ID NO: 17).

AMV-NtADH: In this construct, the viral translational enhancer element 5′ UTR of the AMV (SEQ ID NO: 3) was tandemly stacked with the cellular translational enhancer element 5′ UTR (SEQ ID NO: 4) of the NtADH gene. The elements of this construct were positioned in the following 5′-3′ order: (1) a Cestrum promoter from yellow leaf curling virus (SEQ ID NO: 12; (2) the AMV enhancer element (SEQ ID NO: 3); (3) the NtADH translational enhancer element (SEQ ID NO:4); (4) a 6 bp Soy Kozak sequence (SEQ ID NO: 13); (5) a signal sequence from soybean glycinin seed protein (SEQ ID NO: 14); (6) a reporter comprising the endoglucanase-encoding sequence (SEQ ID NO: 15); (7) an ER retention signal (SEQ ID NO: 16); and (8) a t35s transcription terminator (SEQ ID NO: 17).

TEV-NtADH: In this construct, the viral translational enhancer element 5′ UTR of the Tobacco Etch Virus (TEV); (SEQ ID NO: 2) was tandemly stacked with the cellular translational enhancer element 5′ UTR (SEQ ID NO: 4) of the NtADH gene. The elements of this construct were positioned in the following 5′-3′ order: (1) a Cestrum promoter from yellow leaf curling virus (SEQ ID NO: 12; (2) the TEV enhancer element (SEQ ID NO: 2); (3) the NtADH translational enhancer element (SEQ ID NO: 4); (4) a 6 bp Soy Kozak sequence (SEQ ID NO: 13); (5) a signal sequence from soybean glycinin seed protein (SEQ ID NO: 14); (6) a reporter comprising the endoglucanase-encoding sequence (SEQ ID NO: 15); (7) an ER retention signal (SEQ ID NO: 16); and (8) a t35s transcription terminator (SEQ ID NO: 17).

Ω-ZmADH: In this construct, the viral translational enhancer element 5′ UTR of TMV (SEQ ID NO: 1) was tandemly stacked with the cellular translational element 5′ UTR (SEQ ID NO:7) of the Zea mays alcohol dehydrogenase (ZmADH) gene. The elements of this construct were positioned in the following 5′-3′ order: (1) a Cestrum promoter from yellow leaf curling virus (SEQ ID NO:12; (2) Ω (SEQ ID NO:1); (3) the ZmADH translational enhancer element (SEQ ID NO:7); (4) a 6 bp Soy Kozak sequence (SEQ ID NO:13); (5) a signal sequence from soybean glycinin seed protein (SEQ ID NO:14); (6) a reporter comprising the endoglucanase-encoding sequence (SEQ ID NO:15); (7) an ER retention signal (SEQ ID NO:16); and (8) a t35s transcription terminator (SEQ ID NO:17).

2. Single Constructs

Ω construct: The elements of this construct were positioned in the following 5′-3′ order: (1) a Cestrum promoter from yellow leaf curling virus (SEQ ID NO:12); (2) the Ω enhancer element (SEQ ID NO: 1); (3) a 6 bp Soy Kozak sequence (SEQ ID NO:13); (4) a signal sequence from soybean glycinin seed protein (SEQ ID NO:14); (5) an endoglucanase-encoding sequence (SEQ ID NO:15); (6) an ER retention signal (SEQ ID NO:16); and (7) the t35s transcription terminator (SEQ ID NO:17).

NtADH construct: The elements of this construct were positioned in the following 5′-3′ order: (1) a Cestrum promoter from yellow leaf curling virus (SEQ ID NO:12); (2) the NtADH translational enhancer element (SEQ ID NO:4); (3) a 6 bp Soy Kozak sequence (SEQ ID NO:13); (4) a signal sequence from soybean glycinin seed protein (SEQ ID NO:14); (5) an endoglucanase-encoding sequence (SEQ ID NO:15); (6) an ER retention signal (SEQ ID NO:16); and (7) the t35s transcription terminator (SEQ ID NO:17).

ZmADH construct: The elements of this construct were positioned in the following 5′-3′ order: (1) Cestrum promoter from yellow leaf curling virus (SEQ ID NO:12); (2) the ZmADH translational enhancer element (SEQ ID NO: 7); (3) a 6 bp soy Kozak sequence (SEQ ID NO: 13); (4)) a signal sequence from soybean glycinin seed protein (SEQ ID NO:14); (5) a endoglucanase-encoding sequence (SEQ ID NO:15); (6) an ER retention signal (SEQ ID NO:16); and (7) the t35s transcription terminator (SEQ ID NO:17).

### T0 Tobacco Transformation & Sampling

Tobacco transformation was highly variable in terms of total events recovered and copy number distribution per construct. In addition, several events consistently indicated a mixed copy number eg. the selectable marker was consistently 2-copy and EG was consistently 1-copy.

Tobacco plants were sampled at two time points relative to the date they were transplanted from a transformation culture vessel into soil in the greenhouse. Samples (approximately 3 hole-punches worth) were taken at 13 and 34 days after transplant. 13-day plants had 2-4 leaves. Samples were collected from the youngest leaf. In instances where the youngest leaf was too small, the second youngest leaf was sampled. 34-day plants had entered flowering. At this stage, the lowest (most mature) green leaf was selected for sampling. Tissue samples were analyzed by ELISA and TAQMAN®.

Most events were sampled at both 13 days and 34 days. In some cases however and event was screened in one and not the other. Events which had no expression in the 13 day samples were discarded. Events which produced low expression at 13 days but none at 34 days were not included in the 34 day data set. Finally some plants were missed during the 13 day screen. These plants were not assayed at 13 days but were assayed at 34 days. This is why event numbers are slightly different for some constructs between the 13 day and 34 day data sets.

### T0 Young Tobacco Results

Average expression for 4 out of 5 double enhancers was higher than the enhancer-less control the exception being Ω+ZmHSP101. Ω alone and NtADH+Ω performed poorly, relative to the enhancer-less control, as expected based upon the transient data.

T0 Tobacco 13 Days After Transplant

| | | Average | |
|---|---|---|---|
| | | Expression | |
| | | (ng EG/mg | Standard |
| Enhancer | Events | TP) | Deviation |
| No Enhancer | 9 | 64.8 | 80.7 |
| NtADH | 11 | 113.3 | 48.1 |
| Ω + NtADH | 12 | 110.5 | 61.6 |
| AMV + NtADH | 16 | 87.6 | 59.6 |
| TEV + NtADH | 16 | 256.4 | 143.3 |
| ZmADH | 5 | 129.0 | 104.7 |
| Ω + ZmADH | 11 | 112.8 | 55.7 |
| ZmHSP101 | 20 | 46.2 | 20.6 |
| Ω + ZmHSP101 | 12 | 36.3 | 17.2 |
| Ω | 20 | 51.0 | 24.2 |
| NtADH + Ω | 9 | 28.5 | 22.1 |

### T0 Mature Tobacco Results

Average expression across all constructs was higher in the mature samples as expected. The same trends observed in the young samples were observed in the mature samples with two exceptions. Events with NtADH had significantly lower expression levels than previously observed. This result was confirmed in a second sampling. It is unknown why expression levels dropped. The second exception was Ω+ZmADH which performed much better than the single enhancer control (ZmADH alone).

T0 Tobacco 34 Days after Transplant

| | | Average | |
|---|---|---|---|
| | | Expression | |
| | | (ng EG/mg | Standard |
| Enhancer | Events | TP) | Deviation |
| No Enhancer | 7 | 109.2 | 61.9 |
| NtADH | 12 | 29.5 | 20.8 |
| Ω + NtADH | 14 | 208.8 | 153.4 |
| AMV + NtADH | 16 | 188.1 | 85.5 |
| TEV + NtADH | 16 | 359.3 | 148.5 |
| ZmADH | 4 | 170.5 | 70.9 |
| Ω + ZmADH | 16 | 323.8 | 109.5 |
| ZmHSP101 | 19 | 38.4 | 26.6 |
| Ω + ZmHSP101 | 14 | 55.9 | 37.4 |
| Ω | 20 | 42.9 | 33.8 |
| NtADH + Ω | 5 | 61.3 | 34.3 |

### T1 Tobacco

Up to 11 events from each construct were selected for T1 analysis. In addition to previously screened events, extra events were added for TEV+NtADH. T1 seed from 2 events from TEV+NtADH, which had not been missed during T0 analysis, were analyzed. T1 seed from each event was germinated in a growth chamber. Samples were taken from the largest leaf (approximately 3 hole-punches worth) 23 days after planting. Offspring were analyzed for copy number via TAQMAN®. Only 1-copy and 2-copy T1 plants from each T0 event were assayed via ELISA. An average of all 1-copy sibs for a given event was considered representative of the event. A similar average was taken for 2-copy plants. Event results were averaged by construct to obtain performance data based on enhancer combination.

### T1 Young Tobacco Results

Construct performance was similar when comparing only single copy plants or only double copy plants. Average expression for 4 out of 5 double enhancers was higher than the enhancer-less control the exception being Ω+ZmHSP101 where data was not available. Ω alone performed poorly relative to the enhancer-less control as was observed in the young T0 screen. In this experiment, NtADH+Ωperformed better that the enhancer-less control. The results from this T1 screen were similar to the transient observation for NtADH and Ω+NtADH. The single enhancer increased expression and the addition of the second translational enhancer further boosted expression. This was also observed for AMV and TEV in combination with NtADH. This did not appear to be the case with ZmADH & Ω+ZmADH.

T1 Tobacco 23 Days after Transplant

| | Single Copy | | | Double Copy | | |
|---|---|---|---|---|---|---|
| | | Average | | | Average | |
| | | Expression | | | Expression | |
| | | (ng EG/ | Standard | | (ng EG/mg | Standard |
| Enhancer | Events | mg TP) | Deviation | Events | TP) | Deviation |
| No Enhancer | 2 | 30.4 | 4.2 | 3 | 56.7 | 14.0 |
| NtADH | 10 | 74.9 | 36.5 | 6 | 95.7 | 55.3 |
| Ω + NtADH | 8 | 105.7 | 53.0 | 6 | 122.0 | 66.1 |
| AMV + NtADH | 2 | 203.1 | 8.9 | 4 | 406.5 | 170.2 |
| TEV + NtADH | 3 | 243.2 | 59.7 | 3 | 191.9 | 171.7 |
| ZmADH | 4 | 140.7 | 80.8 | 3 | 190.2 | 118.1 |
| Ω + ZmADH | 10 | 137.1 | 33.8 | 5 | 212.4 | 74.3 |
| ZmHSP101 | | | | | | |
| Ω + ZmHSP101 | 10 | 50.7 | 10.6 | 10 | 98.8 | 30.7 |
| Ω | 10 | 52.9 | 17.6 | 7 | 102.4 | 33.4 |
| NtADH + Ω | 1 | 131.0 | | 1 | 203.9 | |

### Example 4

### Tandemly Stacked Translational Enhancer Elements Enhance Expression in a Maize Stable Expression T0 System

1. Tandem Constructs:

Ω-NtADH construct: In this construct, the viral translational enhancer element 5′ UTR of the Tobacco Mosaic Virus (TMV) (SEQ ID NO: 1) was tandemly stacked with the cellular translational enhancer element 5′ UTR of the NtADH gene (SEQ ID NO: 4). The elements of this construct were positioned in the following 5′-3′ order: (1) a PEPC promoter from Zea mays (SEQ ID NO: 20; (2) the Ω enhancer element (SEQ ID NO: 1); (3) the NtADH translational enhancer element (SEQ ID NO:4); (4) a 6 bp Maize Kozak sequence (SEQ ID NO:13); (5) a gamma zein signal sequence from Zea mays (SEQ ID NO: 21); (6) an monocot optimized endoglucanase-encoding sequence (SEQ ID NO: 23); (7) an ER retention signal (SEQ ID NO: 16); and (8) a PEPC transcription terminator from Zea mays (SEQ ID NO: 25).

AMV-NtADH: In this construct, the viral translational enhancer element 5′ UTR (SEQ ID NO: 2) of AMV gene was tandemly stacked with the cellular translational enhancer element 5′ UTR (SEQ ID NO: 4) of the NtADH gene. The elements of this construct were positioned in the following 5′-3′ order: (1) a PEPC promoter from Zea mays (SEQ ID NO: 20); (2) the AMV enhancer element (SEQ ID NO:2); (3) the NtADH translational enhancer element (SEQ ID NO:4); (4) a 6 bp Maize Kozak sequence (SEQ ID NO: 22); (5) a Gamma Zein Signal (SEQ ID NO: 21); (6) a reporter comprising the monocot optimized endoglucanase-encoding sequence (SEQ ID NO: 23); (7) an ER retention signal (SEQ ID NO: 16); and (8) a PEPC transcription terminator (SEQ ID NO: 25).

TEV-NtADH: In this construct, the viral translational enhancer element 5′ UTR (SEQ ID NO: 2) of the Tobacco Etch Virus (TEV) was tandemly stacked with the cellular translational enhancer element (SEQ ID NO: 4) 5′ UTR of the NtADH gene. The elements of this construct were positioned in the following 5′-3′ order: (1) a PEPC promoter (SEQ ID NO: 20); (2) the TEV enhancer element (SEQ ID NO: 2); (3) the NtADH translational enhancer element (SEQ ID NO: 4); (4) a 6 bp maize Kozak sequence (SEQ ID NO: 22); (5) a Gamma Zein signal sequence (SEQ ID NO: 21); (6) a reporter comprising the monocot optimized endoglucanase-encoding sequence (SEQ ID NO: 24); (7) an ER retention signal (SEQ ID NO:16); and (8) a PEPC transcription terminator (SEQ ID NO: 25).

Ω-ZmADH: In this construct, the viral translational enhancer element 5′ UTR of TMV (Ω) (SEQ ID NO: 1) was tandemly stacked with the cellular translational enhancer element 5′ UTR of the Zea mays alcohol dehydrogenase (ZmADH) gene (SEQ ID NO: 7). The elements of this construct were positioned in the following 5′-3′ order: (1) a PEPC promoter (SEQ ID NO: 20); (2) Ω (SEQ ID NO: 1); (3) the ZmADH translational enhancer element (SEQ ID NO: 7); (4) a 6 bp maize Kozak sequence (SEQ ID NO: 22); (5) a Gamma Zein signal sequence (SEQ ID NO: 21); (6) a reporter comprising a monocot endoglucanase-encoding sequence (SEQ ID NO: 15); (7) an ER retention signal (SEQ ID NO:16); and (8) a PEPC transcription terminator (SEQ ID NO: 25).

2. Single Constructs:

NtADH construct: The elements of this construct were positioned in the following 5′-3′ order: (1) a PEPC promoter (SEQ ID NO: 20); (2) the NtADH translational enhancer element (SEQ ID NO: 4); (3) a 6 bp maize Kozak sequence (SEQ ID NO: 22); (4) Gamma Zein signal sequence (SEQ ID NO: 21); (5) an monocot optimized endoglucanase-encoding sequence (SEQ ID NO: 15); (6) an ER retention signal (SEQ ID NO:16); and (7) the PEPC transcription terminator (SEQ ID NO: 25).

ZmADH construct: The elements of this construct were positioned in the following 5′-3′ order: (1) a PEPC (SEQ ID NO: 20); (2) the ZmADH translational enhancer element (SEQ ID NO: 7); (3) a 6 bp maize Kozak sequence (SEQ ID NO: 22); (4) Gamma Zein signal sequence (SEQ ID NO: 21); (5) a maize optimized endoglucanase-encoding sequence (SEQ ID NO: 23); (6) an ER retention signal (SEQ ID NO: 16); and (7) the PEPC transcription terminator (SEQ ID NO: 25).

### T0 Maize Sampling

Young corn plants were sampled 6 days after transplant from a transformation culture vessel into soil. Plants at this stage had an average of 2-4 leaves. Samples (approximately 3 hole-punches worth) were collected from the youngest visible leaf tip. In instances where the youngest visible leaf tip was too small, the second youngest leaf tip was sampled. All events screened from transformation contained a single copy of the selectable marker and gene of interest and were backbone free as assayed by TAQMAN®. Events were verified as single copy a second time with TAQMAN®. ELISA assays were used to quantify expression.

### T0 Young Maize Results

All double enhancer combinations outperformed the enhancer-less control. All double enhancer combinations also out-performed their associated single enhancer control. ZmADH increased expression over the enhancer-less control and the addition of a second translational enhancer further increased expression.

T0 Corn 6 Days after Transplant

| | | Average | |
|---|---|---|---|
| | | Expression | |
| | | (ng EG/mg | Standard |
| Enhancer | Events | TP) | Deviation |
| No Enhancer | 31 | 92.0 | 39.3 |
| NtADH | 37 | 71.3 | 25.3 |
| Ω + NtADH | 28 | 109.0 | 24.9 |
| AMV + NtADH | 44 | 129.9 | 47.4 |
| TEV + NtADH | 21 | 198.8 | 69.3 |
| ZmADH | 16 | 128.7 | 45.0 |
| Ω + ZmADH | 17 | 161.6 | 77.7 |
| ZmHSP101 | 21 | 82.9 | 30.5 |
| Ω + ZmHSP101 | 29 | 143.6 | 42.5 |

### Example 5

### T1 Maize Results

The T1 maize data did not mirror young T0 data. In the one experiment conducted in T1 maize plants, the results showed that enhancer constructs' reporter gene (gene of interest) expression was lower than or equivalent to the enhancer-less control. It is believed that the plants were too young when sampled. The photosynthetic promoter used (PEPC), was likely not fully active in the very young leaves. Slight variations in leaf emergence at this young stage may have had a profound effect on promoter functionality which likely masked the effect of the enhancers.

These experiments demonstrate the first known examples of enhanced polypeptide production in plants by utilizing polynucleotide constructs having tandemly stacked viral and cellular translational enhancer elements. The order of the tandemly stacked translational enhancer elements was important, as expression increased when the viral translational enhancer element was positioned upstream of the cellular translational enhancer element. This orientation typically increased EG expression by at least 2-fold over that achieved in the absence of any translational enhancer element, and at least 25% over the level of expression that could be achieved with the use of a single cellular translational enhancer element.

The article “a” and “an” are used herein to refer to one or more than one (i.e., to at least one) of the grammatical object of the article. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

As used herein, “about” means within a statistically meaningful range of a value, for example, a stated concentration, length, molecular weight, purity, time, or temperature. Such a range can be within an order of magnitude, typically within 20%, more typically within 10%, and more typically still within 5% of a given value or range. The allowable variation encompassed by “about” will depend upon the particular system under study, and can be readily appreciated by one of skill in the art.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

That which is claimed is:

1. A polynucleotide construct comprising (a) at least one translational enhancer element derived from a virus tandemly stacked with at least one translational enhancer element derived from a cellular gene, and (b) an operably linked polynucleotide encoding a polypeptide of interest.

2. The polynucleotide construct of claim 1, wherein said virus is a plant virus.

3. The polynucleotide construct of claim 2, wherein said virus is an RNA virus.

4. The polynucleotide construct of claim 3, wherein said virus is a member of the Group IV (+)ssRNA viruses, and wherein said translational enhancer element derived from said virus comprises the leader sequence (5′ UTR) of said virus.

5. The polynucleotide construct of claim 4, wherein said virus is a member of the genus Tobamovirus or is a member of a family selected from the group consisting of the Potyviridae, Bromoviridae, and Tombusviridae.

6. The polynucleotide construct of claim 5, wherein said virus is selected from the group consisting of tobacco mosaic virus (TMV), tobacco etch virus (TEV), alfalfa mosaic virus (AMV), and maize necrotic streak virus (MNeSV).

7. The polynucleotide construct of claim 6, wherein said virus is TMV, and wherein said translational enhancer element derived from said TMV comprises the leader sequence set forth in SEQ ID NO: 1 or a functional fragment or variant thereof, wherein said variant has at least 95% sequence identity to the sequence set forth in SEQ ID NO: 1.

8. The polynucleotide construct of claim 6, wherein said virus is TEV, and wherein said translational enhancer element derived from said TEV comprises the leader sequence set forth in SEQ ID NO: 2 or SEQ ID NO: 18, or a functional fragment or variant thereof, wherein said variant has at least 95% sequence identity to the sequence set forth in SEQ ID NO: 2 or SEQ ID NO: 18.

9. The polynucleotide construct of claim 6, wherein said virus is AMV or MNeSV, and wherein said translational enhancer element derived from said AMV or said MNeSV comprises the leader sequence set forth in SEQ ID NO: 3 or SEQ ID NO: 19, respectively, or a functional fragment or variant thereof, wherein said variant has at least 95% sequence identity to the sequence set forth in SEQ ID NO: 3 or SEQ ID NO: 19.

10. The polynucleotide construct of claim 1, wherein said translational enhancer element derived from a cellular gene is a alcohol dehydrogenase gene

11. The polynucleotide construct of claim 10, wherein said alcohol dehydrogenase gene is from a monocot plant or a dicot plant.

12. The polynucleotide construct of claim 10, wherein said alcohol dehydrogenase gene is from tobacco, rice, Arabidopsis, soy or maize.

13. The polynucleotide construct of claim 10, wherein said translational enhancer element derived from said cellular gene comprises the tobacco alcohol dehydrogenase leader sequence set forth in SEQ ID NO: 4, or a functional fragment or variant thereof, wherein said variant has at least 95% sequence identity to the sequence set forth in SEQ ID NO: 4.

14. The polynucleotide construct of claim 10, wherein said translational enhancer element derived from said cellular gene comprises the maize alcohol dehydrogenase leader sequence set forth in SEQ ID NO: 7, or a functional fragment or variant thereof, wherein said variant has at least 95% sequence identity to the sequence set forth in SEQ ID NO: 7.

15. An expression cassette comprising the polynucleotide construct of claim 13.

16. An expression cassette comprising the polynucleotide construct of claim 14.

17. The expression cassette of claim 15, wherein said polynucleotide construct is operably linked to a promoter that is functional in a plant cell, and wherein said promoter is selected from the group consisting of a constitutive promoter, an inducible promoter, and a tissue-specific promoter.

18. The expression cassette of claim 16, wherein said polynucleotide construct is operably linked to a promoter that is functional in a plant cell, and wherein said promoter is selected from the group consisting of a constitutive promoter, an inducible promoter, and a tissue-specific promoter.

19. A plant comprising the polynucleotide construct of claim 1.

20. A plant comprising the polynucleotide construct of claim 17.

21. A plant comprising the polynucleotide construct of claim 18.

22. The plant of claim 19, wherein said polynucleotide construct or said expression cassette is stably integrated into the genome of the plant, and wherein said polynucleotide construct is operably linked to a promoter that is functional in a plant cell.

23. The plant of claim 20, wherein said polynucleotide construct or said expression cassette is stably integrated into the genome of the plant, and wherein said polynucleotide construct is operably linked to a promoter that is functional in a plant cell.

24. The plant of claim 21, wherein said polynucleotide construct or said expression cassette is stably integrated into the genome of the plant, and wherein said polynucleotide construct is operably linked to a promoter that is functional in a plant cell.

25. A cell of the plant of claim 22, wherein said cell comprises said polynucleotide construct or said expression cassette stably integrated into its genome, and wherein said polynucleotide construct is operably linked to a promoter that is functional in a plant cell.

26. A cell of the plant of claim 23, wherein said cell comprises said polynucleotide construct or said expression cassette stably integrated into its genome, and wherein said polynucleotide construct is operably linked to a promoter that is functional in a plant cell.

27. A cell of the plant of claim 24, wherein said cell comprises said polynucleotide construct or said expression cassette stably integrated into its genome, and wherein said polynucleotide construct is operably linked to a promoter that is functional in a plant cell.

28. Seed of the plant of claim 22, wherein said seed comprises said polynucleotide construct or said expression cassette stably integrated into its genome, and wherein said polynucleotide construct is operably linked to a promoter that is functional in a plant cell.

29. Seed of the plant of claim 23, wherein said seed comprises said polynucleotide construct or said expression cassette stably integrated into its genome, and wherein said polynucleotide construct is operably linked to a promoter that is functional in a plant cell.

30. Seed of the plant of claim 24, wherein said seed comprises said polynucleotide construct or said expression cassette stably integrated into its genome, and wherein said polynucleotide construct is operably linked to a promoter that is functional in a plant cell.
